(19) United States
(12) Patent Application Publication
(10) Pub. No.: US 2013/0177921 A1
(43) Pub. Date: Jul. 11, 2013

(54) Detection of Damage to DNA

(71) Applicant: The University of North Carolina at Chapel Hill, Chapel Hill, NC (US)

(72) Inventors: Paul Chastain, Pittsboro, NC (US); David Kaufman, Chapel Hill, NC (US); Stephanie M. Cohen, Durham, NC (US)

(73) Assignee: The University of North Carolina at Chapel Hill, Chapel Hill, NC (US)

(21) Appl. No.: 13/685,427

(22) Filed: Nov. 26, 2012

Related U.S. Application Data
(63) Continuation-in-part of application No. PCT/US2011/037858, filed on May 25, 2011.
(60) Provisional application No. 61/348,168, filed on May 25, 2010.

(51) Int. Cl. C12Q 1/68; G01N 33/58

(52) U.S. Cl. CPC C12Q 1/68; G01N 33/582; G01N 33/58
USPC 435/6.19; 435/6.1; 204/451

(57) ABSTRACT

The present invention relates to methods of assessing damage to cellular DNA including the type, frequency and/or distribution of the DNA damage in the genome. The invention further provides methods of evaluating DNA damage in a cell caused by an agent and/or event as well as methods of determining a subject's prior exposure to an agent and/or event that is known or suspected to cause DNA damage. Further provided are methods of determining whether a subject is at an increased risk for a disease or disorder as a result of cellular DNA damage.

### RELATED APPLICATION INFORMATION

This application is a continuation-in-part application of International Application Serial No. PCT/US2011/037858; Filed: May 25, 2011, which claims the benefit of U.S. Provisional Application No. 61/348,168; filed May 25, 2010, the disclosures of which are incorporated by reference herein in their entireties.

### STATEMENT OF FEDERAL SUPPORT

This invention was made with government support under Grant Nos. CA084493, CA125337, ES010126. ES005948, ES015856 and ES007017 awarded by The National Institutes of Health. The government has certain rights in the invention.

### FIELD OF THE INVENTION

The present invention relates to methods of detecting damage to DNA. In particular, the present invention relates to methods of detecting the frequency, type and/or distribution of DNA damage in a cell, of evaluating DNA damage in a cell caused by an agent and/or event as well as methods of determining a subject's prior exposure to an agent and/or event that is known or suspected to cause DNA damage and/or determining whether a subject is at an increased risk for a disease or disorder as a result of cellular DNA damage.

### BACKGROUND OF THE INVENTION

Reactive oxygen species (ROS) are a class of reactive ions and free radicals generated within cells by oxidative reactions both as products of endogenous metabolism and in response to environmental exposures. Inside the cell, ROS are generated in a variety of ways, as byproducts of energy production in mitochondria, as part of an antimicrobial or antiviral response, and in detoxification reactions carried out by the cytochrome P-450 system. Environmental factors such as chlorinated compounds, radiation, metal ions, barbiturates, phorbol esters, some peroxisome proliferating compounds, and ultraviolet light can also induce the formation of ROS inside the cell. Once formed, ROS can react with macromolecules and lipids. In DNA they create several distinct oxidative DNA damage products: 8-hydroxyguanine (8-oxoG) and apurinic apyrimidinic sites (AP/abasic sites) are the damage products most studied (1, 2). The base excision repair (BER) pathway repairs these DNA base lesions (in addition to the lesions generated by alkylation and deamination). BER includes two major processes, the single-nucleotide (SN)-BER and long-patch (LP)-BER pathways, distinguished by their repair patch size and the enzymes they require. In addition to the formation of AP sites during BER, AP sites form through spontaneous depurinations and depyrimidinations reactions in each cell per day (3, 4).

Normally, the cell's antioxidant defense mechanisms are able to eliminate most of the ROS that are formed and minimize the formation of ROS-induced AP sites. When cells cannot efficiently eliminate ROS, they suffer the consequences of oxidative stress, including increased ROS-induced damage of DNA. The excessive production of ROS and subsequent oxidative stress and cellular damage has been linked to the pathogenesis of many age-related and chronic diseases. These include ischemia/reperfusion injuries (5, 6), Alzheimer's disease (7-9), amyotrophic lateral sclerosis (ALS) (10), Parkinson's disease (11-14), atherosclerosis (15-18), cataract formation (19-22), macular degeneration (23, 24), the aging process (25-28), and cancer (29-32).

There is a need in the art for improved methods of detecting the frequency, type and/or distribution of DNA damage, of evaluating DNA damage caused by an agent and/or event as well as methods of determining a subject's prior exposure to an agent and/or event that is known or suspected to cause DNA damage and/or determining whether a subject is at an increased risk for a disease or disorder as a result of DNA damage.

### SUMMARY OF THE INVENTION

The present invention provides a faster, easier method of evaluating DNA damage, which is amenable to being carried out as a semi-automated or fully automated process. The results achieved with the methods of the invention are similar to those seen with slot blot analysis, which is currently the accepted “gold standard” in the field. The methods of the invention, however, use much fewer cells and DNA, are faster to carry out, and can be used to assess multiple types of damage concurrently or sequentially with a single DNA fiber preparation.

Accordingly, as one aspect, the invention provides a method of assessing DNA damage in a cell, the method comprising:

(a) preparing a DNA fiber from the cell; then

(b) labeling the DNA fiber prepared from the cell with a tag comprising a detectable moiety, wherein the tag comprising the detectable moiety associates with damaged DNA; and

(c) detecting the tag comprising the detectable moiety associated with damaged DNA in the DNA fiber, thereby assessing DNA damage in the cell.

As another aspect, the invention provides a method of assessing DNA damage in a subject or cell, the method comprising:

(a) contacting a cell or DNA prepared therefrom with a tag comprising a detectable moiety, wherein the tag comprising the detectable moiety associates with damaged DNA;

(b) preparing a DNA fiber from the cell or DNA prepared therefrom; and

(c) detecting the tag comprising the detectable moiety associated with damaged DNA in the DNA fiber, thereby assessing DNA damage in the cell.

In a further aspect, the invention provides a method of assessing DNA damage in a cell, the method comprising,

(a) preparing a DNA fiber from the cell;

(b) introducing the DNA fiber into a microfluidic or nanofluidic channel,

(c) establishing a voltage across or through the channel; and

(d) detecting a change in the electrical current across the channel as the DNA moves through the channel, thereby assessing the DNA damage in the cell. In some embodiments, the DNA is tagged and in other embodiments the DNA is not tagged.

In embodiments of the invention, the method is a method of assessing DNA damage following an event that may damage DNA. Optionally, the event comprises exposure to a chemical, an electromagnetic source, ultraviolet radiation, ionizing radiation and/or an agent that causes oxidative damage to DNA.

In representative embodiments, the method is a method of assessing DNA damage following two or more simultaneous and/or sequential events that may damage DNA.

In embodiments of the invention, the DNA fiber is formed from isolated DNA.

Alternatively, in other representative embodiments, the DNA fiber is formed from chromatin.

According to embodiments of the invention, the tag comprising the detectable moiety recognizes a protein that detects and/or repairs damaged DNA.

In embodiments of the invention, the method further comprises labeling the DNA fiber with a reagent that indicates DNA replication, wherein the reagent that indicates DNA replication comprises a detectable moiety that is different from the detectable moiety(ies) used to label the damaged DNA. In embodiments of the invention, the reagent that indicates DNA replication is a nucleotide precursor and/or a reagent that recognizes a replication and/or checkpoint protein. In representative embodiments, the method can further comprise detecting the reagent that indicates DNA damage comprising the detectable moiety.

In embodiments of the invention, the method is carried out on a microscope slide.

In embodiments of the invention, the cell is a cell derived from ectoderm, a cell derived from endoderm, a cell derived from mesoderm, a stern cell, a skin cell, a cell from a pre-cancerous lesion and/or a cancer cell.

Further, in representative embodiments, the cell is from a cell or tissue culture or from a subject (a cell ex vivo).

In embodiments of the invention, when the cell is from a subject, the method can further comprise administering to the subject a reagent that indicates DNA replication, wherein the reagent that indicates DNA replication comprises a detectable moiety that is different from the detectable moiety(ies) used to label the damaged DNA. In representative embodiments, the method can further comprise detecting the reagent that indicates DNA damage comprising the detectable moiety.

In embodiments of the invention, the method further comprises determining whether the subject is at an elevated risk of developing a pre-cancerous or cancerous lesion.

In embodiments of the invention, the method further comprises determining whether the subject is at an elevated risk of developing an age-related and/or chronic disorder such as ischemia/reperfusion injury, Alzheimer's disease, amylotrophic lateral sclerosis. Parkinson's disease, atherosclerosis, cataracts and/or macular degeneration.

As a further option, in representative embodiments, the method further comprises (a) contacting the DNA fiber with a test agent prior to and/or concurrently with labeling the DNA fiber with the tag comprising the detectable moiety that associates with damaged DNA; or (b) contacting the cell or DNA therefrom with a test agent prior to and/or concurrently with contacting the cell or DNA therefrom with the tag comprising the detectable moiety that associates with damaged DNA. In representative embodiments, the test agent is a chemical agent, an electromagnetic agent, ultraviolet radiation, ionizing radiation and/or an agent that causes oxidative damage to DNA.

In embodiments of the invention, the method comprises labeling the DNA fiber with a second tag that associates with a different form of DNA damage than the first tag, and wherein the second tag comprises a second detectable moiety that differs from the first detectable moiety. Optionally, the method can further comprise detecting the second tag comprising the second detectable moiety.

In embodiments of the invention, the method is a quantitative method.

In embodiments of the invention, the method further comprises labeling the DNA fiber with a reagent that associates with DNA, wherein the reagent that associates with DNA comprises a detectable moiety that is different from the detectable moiety(ies) used to label the damaged DNA. Optionally, the method can further comprise detecting the reagent that associates with DNA.

In representative embodiments, the method further comprises labeling the DNA fiber with a reagent that indicates DNA replication, wherein the reagent that indicates DNA replication comprises a detectable moiety that is different from the detectable moiety(ies) used to label the damaged DNA. Optionally, the method can further comprise detecting the reagent that indicates DNA replication comprising the detectable moiety.

In representative embodiments, the method is semi-automated or is automated.

In embodiments of the invention, the detectable moiety is a fluorescent moiety, a histochemically detectable moiety, a colorimetric moiety, a luminescent moiety, a radiolabel and/or an electron-dense moiety.

In further embodiments of the invention, detecting the tag comprising the detectable moiety associated with the damaged DNA comprises imaging the tag comprising the detectable moiety that is associated with the damaged DNA.

In embodiments of the invention, detecting the tag comprising the detectable moiety associated with damaged DNA is carried out using a computer-based method.

According to representative embodiments of the invention, the DNA damage comprises oxidative damage, photolesions, bulky adducts, protein-DNA crosslinks, DNA crosslinks, single-stranded DNA breaks and/or double-stranded DNA breaks. As non-limiting examples, the oxidative, damage can comprise apurinic/apyrimidinic (AP/abasic) sites, 8-hydroxyguanine (8-oxo-dG) sites 2,6-diamino-4-hydroxy-5-formamidopyrimidine (Fapy-Gua), 4,6-diamino-5-formamido-pyrimidine (Fapy-Ade) and/or 7,8-dihydro-8-oxoaclenine (8-oxoaclenine). As further non-limiting examples, the photolesions can comprise cyclobutane pyrimidine dimers (CPD), [6-4]pyrimidine-pyrimidone photoproduct ([6-4]PPs) and/or Dewar isomer of 6-4PPs (Dewar PPs).

In representative embodiments, the method is practiced to assess the type, amount and/or distribution of DNA damage.

In embodiments of the invention. DNA damage within specific regions of the genome is assessed, optionally by fluorescent in situ hybridization (FISH).

In embodiments of the invention, the tag comprising the detectable moiety is an aldehyde reactive probe that recognizes AP sites comprising a detectable moiety, for example, biotin or a fluorescent moiety.

In further representative embodiments, the DNA fiber is prepared in a microfluidic or nanofluidic device. A microfluidic or nanofluidic device can comprise one or many parallel identical channels.

In embodiments of the invention, detecting the tag comprising the detectable moiety associated with damaged DNA is carried out in a microfluidic or nanofluidic device.

These and other aspects of the invention are addressed in more detail in the description of the invention set forth below.

### BRIEF DESCRIPTION OF THE DRAWINGS

FIG. 1 shows detection of AP sites in DNA fiber spreads. Composite image of DNA fibers stained with YOYO-1 green fluorescent dye. AP sites (white arrows) were tagged with biotin using ARP and detected with a red fluorescent anti-biotin antibody. The scale bar provides a measure of the length of DNA fibers in bp.

FIG. 2 shows the number of AP sites in DNA from cells under typical tissue culture conditions or exposed to 20 μM H₂O₂. The number of AP sites per 10⁶ nt determined by slot blot (bars at left) and fiber spread (bars at right) analysis is shown for cells under normal culture conditions and after exposure to 20 μM hydrogen peroxide. The average values are listed at the top of the bars. The slot blot average was determined from 3 independent experiments while the fiber analysis values were determined from analysis of six different slides.

FIG. 3 shows the number of AP sites in areas undergoing replication. Comparison between the average number of AP sites found in replicating DNA fibers before and after exposure to 20 μM H₂O₂. In these experiments only DNA fibers with replicating DNA were quantified. The average values are listed at the top of the bars. The fiber analysis values were determined from analysis of 3 slides.

FIG. 4 shows detection of AP sites in areas undergoing replication in DNA fiber spreads. This is a composite image of multiple DNA fibers containing AP sites and areas undergoing replication. Fiber spreads were prepared from cells that were pulsed with IdU (red fluorescence) for 10 min, exposed to H₂O₂ and then pulsed with CIdU (green fluorescence) for 20 min. IdU and CIdU were identified as described in Example 1. AP sites were tagged with biotin using ARP and the biotin identified using a blue fluorescent antibody. For ease of viewing, the blue signal corresponding to AP sites was electronically changed into white.

FIG. 5 shows a schematic of the detection of DNA damage and areas undergoing replication in DNA.

FIG. 6 shows detection of CPDs in CIdU tracks. CIdU (green) and CPDs (red) were detected in DNA fiber spreads generated from cells that were irradiated with 1 J/m2 UVC. Inset: areas undergoing replication and CPD sites at higher magnification. White arrows indicate the locations of CPDs within the CIdU tracks. Bar equals 62 kilobases (Kb).

FIG. 7 shows a schematic of chromatin fiber preparation and using chromatin fibers to detect DNA damage, repair proteins and checkpoint proteins.

FIG. 8 shows the distribution of DNA damage and repair proteins on chromatin fibers. Normal human fibroblasts (NHF1) cells were treated with 50 μM H₂O₂ for 30 minutes before collection. The distribution of 8-OHdG (red) and 8-oxoguanine-Glycosylase (OGG1, blue) is shown. YOYO-1 (green) was used to counterstain DNA. Bars ˜25 μm (−400 kb, bottom right of each panel).

FIGS. 9A-9B shows the distribution of DNA damage and repair proteins on chromatin fibers. Fig. A. NHF1 cells were treated with 5 J/m² of UVC. DNA was heat denatured to allow for immunofluorescent staining of cyclobutane pyrimidine dimers (CPDs) while retaining chromatin proteins. The distribution of CPDs (blue) on chromatin fibers (H3 staining, red) is shown. Fig. B. As seen with the DNA fibers, once a CPD forms, there is an increased chance of a second CPD forming adjacent to the original CPD. Bars ˜25 μm (˜400 kb, bottom right of each panel).

FIGS. 10A-10B shows detection of collapsed replication forks and DNA double strand breaks in chromatin fibers. Fig. A. NHF1 cells were pulsed with EdU, a thymidine analogue, for 30 minutes prior to being irradiated with 10 J/m² and then collected 15 minutes afterwards. ATRIP is one of the initial proteins that “sense” stalled replication forks. Its colocalization at the EdU (replication) track at the right side (arrow) indicates that that fork stalled due to encountering a DNA lesion (UV damage), Fig. B. NHF1 cells were pulsed with EdU for 30 minutes prior to being irradiated with 20 J/m² and then collected 2 h later. 53bp1 is a protein that is associated with double-strand breaks. Its location at the edge of several EdU tracks suggests those replication forks have collapsed, destabilized, and caused a double-strand break to form. Bars ˜25 μm (˜400 kb, bottom right of each panel).

FIG. 11 shows cyclobutane pyrimidine dimers (CPDs) deposited onto DNA at various fluencies of UVC.

FIG. 12 shows identification of DNA damage sites in fiber tracks determined by software developed for the present invention, Arrows point to DNA Damage identified in a DNA fiber and depicted as a white dot in the outline. Numbers indicate the fiber trace number.

FIG. 13. shows the design of experiments to assess Dox Induced DNA damage in MCF7 cells used as surrogate circulating tumor cells (CTCs). MCF7 cells were exposed to Doxorubicin (Dox) in culture (1) or exposed to Dox in culture and then processed using the Mag Sweeper Oft MCF7 cells processed identically but not treated with Dox served as controls in each case. Cells were next were recovered, put onto slides and lysed and DNA extended

FIG. 14 shows dox-Induced DNA damage in surrogate CTCs. MCF7 cells were exposed to Doxorubicin (Dox) alone (first three bars) or exposed to Dox first then isolated using Mag Sweeper (fourth, filth, sixth bars). Thereafter. DNA was extended on slides following cell lysis and DNA and sites of DNA damage were stained with fluorescent probes Images of extended fluorescently-labeled DNA fibers were obtained and DNA content and DNA damage site number were determined by computer analysis of the images. Results of the analysis of DNA damage in the bars is reported with the ordinate of the graph scaled as the number of DNA damage sites, in this case AP sites, per 10⁶ nucleotides in length

FIGS. 15A-15B demonstrate that CTCs can be found at each stage of breast cancer (FIG. 15A) and in different carcinomas (FIG. 15B). Data derived from or used from FIG. 15A (2) and FIG. 15B (1), respectively.

FIG. 16 provides an exemplary schematic showing detection of DNA and adducted DNA by resistance or impedance change.

### DETAILED DESCRIPTION OF THE INVENTION

The present invention will now be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference herein in their entirety.

As used in the description of the invention and the appended claims, the singular forms “a,” “an” and “the” are intended to include the plural forms as well, unless the context clearly indicates otherwise.

The term “about,” as used herein when referring to a measurable value such as nucleotide bases or basepairs, time, temperature, and the like, is meant to encompass variations of ±20%, ±10%, ±5%, ±1%, ±0.5%, or even ±0.1% of the specified amount.

Numerical ranges as described herein are intended to be inclusive unless the context indicates otherwise. For example, the numerical range of “1 to 10” or “1-10” is intended to be inclusive of the values 1 and 10.

As used herein, “and/or” refers to and encompasses any and all possible combinations of one or more of the associated listed items, as well as the lack of combinations when interpreted in the alternative (“or”).

The features of the invention described herein can be used in any combination. Moreover, the present invention also contemplates that in some embodiments of the invention, any feature or combination of features set forth herein can be excluded or omitted.

As used herein, the term “assess,” “assesses,” or “assessing” DNA damage, DNA replication, DNA repair, DNA checkpoint proteins (and like terms) indicates an evaluation, detection, determination and/or measurement of the type, frequency and/or genomic distribution of DNA damage, DNA replication, DNA repair and DNA checkpoint proteins respectively.

As used herein, a tag comprising a detectable moiety that “associates with damaged DNA” or is “associated with damaged DNA” (and similar terms) indicates that the tag comprising the detectable moiety binds to and/or intercalates into the damaged site of the DNA. Generally, the tag associates preferentially with damaged DNA, although not necessarily exclusively, as compared with intact or undamaged DNA. In representative embodiments, the tag comprising the detectable moiety that “associates with” or is “associated with” damaged DNA recognizes a protein that detects and/or repairs damaged DNA. For example, the tag comprising the detectable moiety can comprise an antibody that specifically binds to a protein that detects and/or repairs damaged DNA (e.g., 8-oxoguanine glycosylase (OGG1), ATRIP, phospho-RPA and/or 53bp1).

By “consisting essentially of” as used herein, it is meant that the indicated subject matter does not include any other material elements (i.e., elements that materially impact the recited subject matter). Thus, the term “consisting essentially of” is not to be interpreted as “comprising.”

Unless one term or the other is expressly indicated, the term “DNA fiber” as used herein encompasses both DNA fibers formed from isolated DNA (e.g., the protein components of chromatin and other proteins associated with genomic DNA are substantially or completely removed, for example, at least about 50%, 60%, 70%, 80%, 90% or more are removed) and DNA fibers formed from chromatin (e.g., the protein components of chromatin including histones, enzymes, transcription factors, and the like are substantially retained, for example, at least about 50%, 60%, 70%, 80%, 90% or more are retained).

As used herein, the term “elevate,” “elevates” or “elevating” and similar terms as well as the term “increase,” “increases” or “increasing” and similar terms refers to an increase or augmentation, for example, of at least about 20%, 30%, 40%, 50%, 60%, 70%, 80%, 90%, 100%, 125%, 150%, 200%, 250%, 300%, 400%, 500% or more. In embodiments of the invention, the degree of elevation or increase is relative to a suitable control, e.g., an average, mean and/or median value based on evaluation of a population, which is optionally matched for age, gender and/or race.

Unless the context indicates otherwise, the term “label,” “labels” or “labeling” the DNA fiber (and similar terms) can refer to labeling the DNA fiber directly, e.g., by contacting the DNA fiber with a tag comprising a detectable moiety that recognizes DNA damage, a reagent comprising a detectable moiety that indicates DNA replication and/or a reagent that comprises a detectable moiety that associates with DNA (i.e., total DNA). As another option, a cell or DNA prepared therefrom can be contacted with the tag(s) and/or reagent(s) comprising the detectable moiety prior to and/or concurrently with preparing the DNA fiber where the resulting DNA fiber is labeled with the tag(s) and/or reagent(s) comprising the detectable moiety.

A “reagent that associates with DNA,” “reagent associated with DNA, “reagent that associates with total DNA,” or “reagent associated with total DNA” (and similar terms) indicates that the reagent binds to and/or intercalates into DNA (e.g., a DNA stain). Such reagents include without limitation YOYO-1 (Invitrogen), DAPI phenylindole) and/ore Hoechst stain (e.g., Hoechst 33258 or Hoechst 33342), which are fluorescent stains that bind strongly to DNA.

As used herein, a “reagent that indicates DNA replication” or a “reagent that is an indicator of DNA replication” and similar terms refer to a reagent that is a marker (e.g., can assess or detect) areas of DNA replication. Nonlimiting examples of suitable reagents that indicate DNA replication include nucleotides and/or a reagent (e.g., an antibody) that recognizes a replication protein and/or a checkpoint protein.

A “subject” as used herein encompasses a subject from any species, including vertebrates and/or invertebrates as well as plants. Further, subjects can be eukaryote and/or prokaryote (e.g., bacterial) species. In representative embodiments, the subject is an avian or mammalian subject, mammalian subjects including but not limited to humans, non-human primates (e.g., monkeys, baboons, and chimpanzees), dogs, cats, goats, horses, pigs, cattle, sheep, and the like, and laboratory animals (e.g., rats, mice, gerbils, hamsters, and the like). Avian subjects include chickens, ducks, turkeys, geese, quails and birds get as pets (e.g., parakeets, parrots, macaws, and the like). Suitable subjects include both males and females and subjects of all ages including embryonic (e.g., in utero or in ovo), infant, juvenile, adolescent, adult and geriatric subjects. In embodiments of the invention, the subject is not a human embryonic subject.

As a first aspect, the present invention provides a method of assessing DNA damage in a cell, the method comprising: (a) preparing a DNA fiber from the cell; (b) labeling the DNA fiber prepared from the cell with a tag comprising a detectable moiety, wherein the tag comprising the detectable moiety associates with damaged DNA; and (c) detecting the tag comprising the detectable moiety associated with damaged DNA in the DNA fiber, thereby assessing DNA damage in the cell. The tag comprising the detectable moiety is thus a marker of DNA damage and correlates with the presence of DNA damage in the DNA fiber.

The inventors have found that it is generally more efficient to label the damaged DNA after preparing the fibers. However, the damaged DNA can alternatively be labeled prior to fiber formation. Accordingly, the invention also contemplates a method of assessing DNA damage in a cell, the method comprising: (a) contacting a cell or DNA prepared therefrom with a tag comprising a detectable moiety, wherein the tag comprising the detectable moiety associates with damaged DNA; (b) preparing a DNA fiber from the cellular DNA; (c) detecting the tag comprising the detectable moiety associated with damaged DNA in the DNA fiber, thereby assessing DNA damage in the cell. Again, the tag comprising the detectable moiety is functioning as a marker of DNA damage and correlates with the presence of DNA damage in the DNA fiber.

As a further aspect, the present invention provides a method of assessing DNA damage in a cell, the method comprising, (a) preparing a DNA fiber from the cell; (b) introducing the DNA fiber into a microfluidic or nanofluidic channel, wherein introduction of a DNA fiber into a microfluidic or nanofluidic channel linearizes the DNA (c) establishing a voltage across or through the channel; and (d) detecting a change in the electrical current across the channel as the DNA moves through the channel, thereby assessing the DNA damage in the cell. In some aspects, establishing a voltage across (transverse) or through (parallel) a channel comprises using at least one pair of electrodes e.g., one pair, two pairs, three pairs, four pairs, five pairs, six pairs, seven pairs, or more) to assess DNA damage. In some particular embodiments, establishing a voltage across (transverse) or through (parallel) to a channel comprises using two pairs of electrodes to assess DNA damage. As used herein “establishing a voltage” means adjusting a voltage such that the desired voltage is achieved in the channel or channels of a microfluidic or nanofluidic device.

Electrodes can be placed at different points along the fluidic pathway. Thus, for example, the electrodes may be transverse such that aligned between the electrodes is substantially perpendicular (e.g., transverse) to the direction of fluid flow. Also in a spaced apart manner, the electrodes can be placed along the direction of fluid flow such that aligned between the electrodes is substantially parallel to the direction of fluid flow. Thus, some embodiments of the invention allow the electrodes to be positioned on the same side of a channel, on the bottom of a channel, or along different surfaces that define a channel. In some particular embodiments, the electrical current of two pairs of electrodes that are placed along the direction of the fluid flow (parallel) can be oriented in the opposite direction of one another so that any interference can be reduced or eliminated.

Accordingly, the electrodes of a pair of electrodes for assessing DNA damage can be transverse or parallel with the fluid flow, thereby being able to detect the changes in current as the DNA flows between the pair of electrodes.

Thus, in representative embodiments, the electrodes are closely spaced along the line of flow (transverse) or separated in the direction of (parallel with) the flow of the carrier fluid through a narrow channel where the cross-sectional area of the channel is only slightly larger than the cross-sectional area of a DNA fiber or a DNA fiber with damage. In some embodiments, the cross-section of the channel can be about 2 nm to 1000 nm. Thus, in some aspects, the cross section of the channel can be about 2 nm, 3, nm, 4 nm, 5 nm, 6 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 55 nm, 60 nm, 65 nm, 79 nm, 75 nm, 80 nm, 85 nm, 90 nm, 95 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, 500 nm, 525 nm, 550 nm, 575 nm, 600 nm, 625 nm, 650 nm, 675 nm, 700 nm, 725 nm, 750 nm, 775 nm, 800 nm, 825 nm, 850 nm, 875 nm, 900 nm, 925 nm, 950 nm, 975 nm, 1000 nm, and the like, and/or any range therein.

In some embodiments, the distance between the electrodes of a pair of electrodes can be about 2 nm to about 1000 nm. Thus, the distance between the electrodes of a pair of electrodes can be, for example, about 2 nm, 3, nm, 4 nm, 5 nm, 6 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm n, 36 nm n, 37 nm n, 38 nm, 39 nm, 40 nm, 41 nm, 42 nm, 43 nm, 44 nm, 45 nm, 46 nm, 47 nm, 48 nm, 49 nm, 50 nm, 55 nm, 60 nm, 65 nm, 79 nm, 75 nm, 80 nm, 85 nm, 90 nm, 95 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, 500 nm, 525 nm, 550 nm, 575 nm, 600 nm, 625 nm, 650 nm, 675 nm, 700 nm, 725 nm, 750 nm, 775 nm, 800 nm, 825 nm, 850 nm, 875 nm, 900 nm, 925 nm, 950 nm, 975 nm, 1000 nm, and the like, and/or any range therein

In other embodiments, the distance between each pair of electrodes used for assessing DNA damage can be between about 10 μM to about 1000 μM. Accordingly, in some embodiments, the distance between one pair of electrodes and another pair of electrodes used for assessing DNA damage can be from about 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 51 μM, 52 μM, 53 μM, 54 μM, 55 μM, 56 μM, 57 μM, 58 μM, 59 μM, 60 μM, 61 μM, 62 pμ, 63 μM, 64 pμ, 65 μM, 66 μM, 67 μM, 68 μM, 69 μM, 70 μM, 71 μM, 72 μM, 73 μM, 74 μM, 75 μM, 76 μM, 77 μM, 78 μM, 79 μM, 80 μM, 81 μM, 82 μM, 83 μM, 84 μM, 85 μM, 86 μM, 87 μM, 88 μM, 89 μM, 90 μM, 91 μM, 92 pμ, 93 μM, 94 μM, 95 μM, 96 μM, 97 μM, 98 pμ, 99 μM 100 μM, 125 μM, 150 μM, 175 μM, 200 pμ, 225 pμ, 250 μM, 275 μM, 300 μM, 325 μM, 350 μM, 375 μM, 400 μM, 425 μM, 450 μM, 475 μM, 500 μM, 525 μM, 550 μM, 575 μM, 600 pμ, 625 μM, 650 μM, 675 μM, 700 μM, 725 μM, 750 μM, 775 μM, 800 μM, 825 μM, 850 μM, 875 μM, 900 μM, 925 μM, 950 μM, 975 μM, 1000 μM and the like, and/or any range therein.

The electrical current between the pair of electrodes can be from about 0.5V to 1000V. Thus, in some embodiments, the voltage is about 0.5, 1, 1.5, 2, 2.5, 3, 3.5, 4, 4.5, 5, 5.5, 6, 6, 5, 7, 7.5, 8, 85, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, 200, 225, 250, 275, 300, 325, 350, 400, 425, 450, 475, 500, 525, 550, 575, 600, 625, 650, 675, 700, 725, 750, 775, 800, 825, 850, 875, 900, 925, 950, 975, 1000, and the like, and/or any range therein.

Any electrode useful with the invention can be used. Some non-limiting examples of electrodes useful with the present invention can include carbon, copper, gold, silver, indium tin oxide, platinum, titanium and/or a conductive polymer,

As described herein, assessing DNA damage comprises detecting a change in the electrical current that is applied across the microfluidic or nanofluidic channel. In representative embodiments, detecting changes in the electrical current comprises detecting changes impedance or resistance of the current. Thus, if an AC current is applied, changes in impedance can be used to detect DNA damage and/or if a DC current is applied, changes in resistance can be used to detect DNA damage.

In some aspects of the invention, the DNA is moved through the channel using micro or nanofluidics as is known in the art. Thus, in some embodiments, movement of the DNA through the microfluidic or nanofluidic device can be via a pumping mechanism. In additional embodiments, a biasing potential that is different from the current applied across the channel from side to side (transverse) or parallel to the fluid flow can be used.

In some particular embodiments, the carrier fluid for the DNA in a microfluidic or nanofluidic device comprises an electrolytic solution (e.g., ionic salt). Non-limiting examples of electrolytic solutions includes sodium chloride (NaCl), potassium chloride (KCl) or phosphate buffered saline. The concentrations of such electrolytic solutions can be about 25 mM to about 500 mM (ionic content). Thus, in some embodiments, the electrolytic solution can be about 25 mM, 30 mM, 35 mM, 40 mM, 45 mM, 50 mM, 55 mM, 60 mM, 65 mM, 70 in M, 75 mM, 80 in M, 85 mM, 90 mM, 95 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, 310 mM, 320 mM, 330 mM, 340 mM, 350 mM, 360 mM, 370 mM, 380 mM, 390 mM, 400 mM, 410 mM, 420 mM, 430 mM, 440 mM, 450 mM, 460 mM, 470 mM, 480 mM, 490 mM, 500 mM and the like, or any range therein. In some particular embodiments, the carrier fluid comprises NaCl.

Thus, in some embodiments of the invention in which microfluidics and/or nanofluidics and at least one pair of electrodes as is used to detect DNA damage as described herein, several factors to be considered include but are not limited (1) the cross-section of the microfluidic/nanofluidic channel (2 nm to 1000 nuM), (2) the voltage between electrodes (0.5 to 1000V), (3) the length of channel (20 nm to 1 mm); (4) the distance between a pair of electrodes and between electrode pairs and/or (5) the ionic content of the carrier fluid.

The invention can be practiced with any suitable cell, which can further be derived from any subject (subjects are as described herein). In representative embodiments, the cell is a mammalian cell and is derived from one of the three primary germ layers (i.e., is derived from ectoderm, endoderm and/or mesoderm), is a stern cell, a skin cell (e.g., an epidermal skin cell, including without limitation a Merkel cell, a melanocyte, a keratinocyte, a dermal dendritic cell, a fibroblast and/or a Langerhans cell), a cell from a pre-cancerous lesion and/or a cancer cell, including a tumor cell (e.g., a skin cancer cell such as a melanoma cell, a basal cell carcinoma cell, a cutaneous squamous cell carcinoma, an actinic keratosis cell, a solar keratosis cell; a colon cancer cell; a cervical cancer cell; a uterine cancer cell; a vaginal cancer cell; a breast cancer cell; a leukemia cell; lymphoma cell; a lung cancer cell; a prostate cancer cell; a brain cancer cell; a kidney clear cell carcinoma cell; an ovarian cancer cell; and the like). Cells according to the present invention also include zygotes (ova and/or sperm) and/or embryonic cells. Further, skin cells can come from any layer of the skin including the epidermis, dermis and/or hypodermis. In embodiments of the invention, the cell is not a human zygote and/or embryonic cell.

In embodiments of the invention, cells can be pre-sorted in a flow cytometer. For example, specific populations of skin cells or tumor cells can be evaluated in this way.

The cell can be from a cell, tissue and/or organ culture in vitro, for example, a primary cell culture or a culture of an immortalized cell line. Alternatively, the cell can be ex vivo from a subject (e.g., without prior culturing). In representative embodiments, the methods of the invention can be practiced with relatively few cells (e.g., about 50 or less, 100 or less, 200 or less, 500 or less, 1000 or less cells, 2000 or less cells, 3000 or less cells, 4000 or less cells, 5000 or less cells, 8000 or less cells, 10,000 or less cells, 12,000 or less cells or 20,000 or less cells). In fact, applying an adhesive strip to the skin (or other site, such as the colon, the mouth, the vagina, the cervix, the uterus, the nasal cavity, and the like) of a subject and then removing the adhesive strip will generally provide a large enough sample of cells to practice the inventive methods. This contrasts with conventional methods in which microgram quantities of cells are used to evaluate each type of DNA damage.

In embodiments of the invention, the cell is exposed to the DNA damaging agent in vivo in a subject and is then removed for analysis of DNA damage, DNA repair proteins. DNA replication, and the like. Pregnant subjects can be used, and the embryo harvested for preparation of DNA fibers. In addition, the subject can be administered a reagent that is an indicator of (i.e., assesses) DNA replication, DNA damage, and the like by any suitable mode of delivery, e.g., intraperitoneal administration, intramuscular administration, intravenous administration, and the like. Tissues, organs and/or cells can be harvested and processed as described herein for the preparation of DNA fibers to detect DNA damage, DNA repair proteins, DNA replication and/or checkpoint proteins.

In representative embodiments, the methods of the invention are practiced to assess DNA damage in a cell following an event that may damage DNA. For example, the event may be (but is not necessarily) one that is known or suspected of causing DNA damage. To illustrate, the invention can be employed to assess DNA damage in a cell following an event including without limitation a chemical exposure, a radiation exposure, a physical stress and/or an electromagnetic exposure. For example, following a chemical or radiation leak, a cell from a subject can be assessed to determine whether the subject has been exposed to the chemical or radiation with resulting DNA damage.

In exemplary embodiments, the event comprises exposure to ultraviolet radiation (e.g., UVA, UVB and/or UVC), ionizing radiation, x-rays and/or gamma rays.

The event can further comprise exposure to hydrolysis, thermal disruption, a plant toxin, a mutagenic chemical (e.g., an aromatic compound that acts as a DNA intercalating agent, a chemotherapeutic agent) and/or a virus.

In other illustrative embodiments, the event comprises exposure to an agent that causes oxidative damage. For example, the agent that causes oxidative damage can comprise a reactive ion or free radical generated by an oxidative reaction. These agents can arise due to endogenous metabolism and/or in response to an environmental exposure. Inside the cell, ROS are generated in a variety of ways, including without limitation, as byproducts of energy production in the mitochondria, as part of an antimicrobial or antiviral response, and/or in detoxification reactions carried out by the cytochrome P-450 system. Environmental factors include without limitation exposure to a chlorinated compound, radiation, a metal ion, a barbiturate, a phorbol ester, a peroxisome proliferating compound and/or ultraviolet light.

In representative embodiments, the method is a method of assessing DNA damage following two or more simultaneous and/or sequential events that may damage DNA (e.g., simultaneous or sequential exposure to ultraviolet radiation and sunscreen or other chemical substance such as a medication, caffeine and/or vitamin D). For example, according to this embodiment, the combination of events can be evaluated to determine whether the combination exacerbates the risks (e.g., has an additive and/or synergistic effect) or if there is a protective effect from the combination (e.g., sunscreen, caffeine and/or vitamin D may protect against ultraviolet damage to DNA).

The methods of the invention can optionally comprise exposing the cell to a test agent or event prior to labeling the DNA fiber with the tag comprising the detectable moiety. According to this embodiment, the invention can be used to evaluate the propensity of the test agent or event to cause and/or protect the cell from DNA damage, e.g., to screen the agent for safety and/or for protective effects. In some embodiments, the methods of the invention can be used to determine a cell's sensitivity or resistance to a test agent such as a chemotherapeutic agent. Optionally, the cell is exposed to the test agent or event prior to preparing the DNA fiber from the cell in vitro, ex vivo or in vivo. Alternatively, the DNA fiber is first prepared and the DNA fiber is then exposed to the test agent or event.

The test agent or event can comprise any agent or event as described above. In embodiments of the invention, the test agent or event comprises exposure to a dermatological agent (e.g., sunscreen, a moisturizer, a topical medication, a cosmetic, a fragrance, and the like) or a chemotherapeutic agent (e.g., a platinum drug such as cisplatin or carboplatin, a PARP inhibitor, and the like).

The test agent can further be any hazardous chemical, for example as listed in the United States Environmental Protection Agency list of Hazardous Materials.

In embodiments of the invention, the cell and/or DNA fiber is exposed to a test agent prior to, concurrently and/or after exposing the cell and/or DNA fiber to a source known to cause DNA damage; in this way, it can be determined whether the test agent has a protective effect and/or acts in an additive or synergistic fashion to enhance DNA damage caused by the known source. In embodiments of the invention, the cell and/or DNA fiber can be exposed to the test agent and known source within about two weeks or less of each other, within about 1 week or less of each other, within about 4 days or less of each other, within about 3 days or less of each other, within about 2 days or less of each other, within about 1 day or less of each other and/or within about 18 hours or less of each other, or can be exposed concurrently to the test agent and known source of DNA damage.

As used herein, “concurrently” (and similar terms) means within minutes or hours (e.g., about 12 hours or less, 9 hours or less, 6 hours or less, 4 hours or less, 3 hours or less, 2 hours or less, 1 hour or less, 45 minutes or less, 30 minutes or less, 20 minutes or less, 10 minutes or less, 5 minutes or less, 4 minutes or less, 3 minutes or less, 2 minutes or less, or 1 minute or less).

As another aspect, the methods of the invention can be practiced to evaluate a subject's previous exposure to an agent(s) that causes DNA damage. According to this embodiment, the method can further comprise determining whether the subject is at an elevated risk of developing a disease or disorder, such as cancer. The type, frequency and/or distribution of DNA damage can be assessed to determine whether the subject has an elevated risk of developing the disease or disorder based on correlations with the type, amount and/or distribution of DNA damage and the risk of developing the disease or disorder.

The invention also contemplates methods of determining a correlation between DNA damage (e.g., type, frequency and/or genomic distribution) and a disease or disorder. In embodiments of the invention, a correlation is made between the type and/or frequency of DNA damage in certain genomic regions (e.g., marker genes such as oncogenes and/or tumor, suppressor genes) and a disease or disorder. Marker genes include without limitation genes that are commonly mutated in a number of skin cancers and other cancers, for example, CDKN2A, p16 (InK4a), p14 (ARP), p53, H-Ras, K-Ras, N-Ras, MYC, GLI1, ABL, APC, BRCA1, BRCA2, SMH2, PTCH, RB, TP53, PTEN, Nrf2, and the like.

The disease or disorder can be any disease or disorder that is associated with an increase in DNA damage including without limitation a precancerous or cancerous lesion (e.g., leukemia, lymphoma, breast cancer, lung cancer, colon cancer, prostate cancer, brain cancer, kidney clear cell carcinoma, ovarian cancer, uterine cancer, cervical cancer and skin cancer such as melanoma, basal cell carcinoma, cutaneous squamous cell carcinoma, actinic keratosis, solar keratosis), an age-related and/or chronic disorder such as ischemia/reperfusion injury, Alzheimer's disease, amylotrophic lateral sclerosis. Parkinson's disease, atherosclerosis, cataracts and/or macular degeneration. Diseases or disorders associated with oxidative stress include without limitation: diseases or disorders of the gastrointestinal tract (e.g., diabetes, pancreatitis, liver damage, and leaky gut syndrome), diseases or disorders of the brain and nervous system (e.g., Parkinson's disease, Alzheimer's disease, hypertension and multiple sclerosis), diseases or disorders of the heart and blood vessels (e.g., atherosclerosis, coronary thrombosis), diseases or disorders of the lungs (e.g., asthma, emphysema, chronic obstructive pulmonary disease), diseases or disorders of the eyes (e.g., cataracts, retinopathy, macular degeneration), diseases or disorders of the joints (e.g., rheumatoid arthritis), diseases or disorders of the kidneys (e.g., glomerulonephritis) and diseases and disorders of the skin (e.g., “age spots”, vitiligo, wrinkles) as well as accelerated aging, autoimmune diseases (e.g., lupus), inflammatory states and HIV/AIDS.

The invention can be used to detect any type of DNA damage, including without limitation oxidative damage, photolesions, bulky adducts, protein-DNA crosslinks, DNA crosslinks, single-stranded DNA breaks and/or double-stranded DNA breaks.

Oxidative damage includes without limitation apurinic/apyrimidinic (AP/abasic) sites, 8-hydroxyguanine (8-oxo-dG) sites, 2,6-diamino-4-hydroxy-5-formamidopyrimidine (Fapy-Gua), 4,6-diamino-5-formamido-pyrimidine (Fapy-Ade) and/or (to a smaller extent) 7,8-dihydro-8-oxoadenine (8-oxoadenine). Examples of photolesions include without limitation cyclobutane pyrimidine dimers (CPD), [6-4]pyrimidine-pyrimidone photoproduct ([6-4]PPs) and/or Dewar isomer of 0-4PPs (Dewar PPs).

In embodiments of the invention, the DNA damage comprises single and/or double-stranded breaks in the DNA fibers. In particular embodiments of the invention, this aspect of the invention is carried out with chromatin fibers, Single- and double-stranded breaks in DNA can be measured by any method known in the art, e.g., by using a peptide or protein (e.g., an antibody) that recognize the break. Alternatively, the presence of specific chromatin modifications that are indicative of chromatin breaks (e.g., the presence of a protein(s) that detects and/or repairs DNA breaks) can be evaluated. For example, the presence of 53bp1 is a marker for double-stranded DNA breaks.

The tag that associates with the damaged DNA can be any suitable molecule that recognizes the damaged DNA. In representative embodiments, the tag is a small molecule, a peptide or a protein. Optionally, the tag is an antibody that specifically recognizes the damaged DNA. A number of antibodies that specifically recognize different forms of DNA damage are readily available, and others can be prepared using known procedures. Antibodies include polyclonal and monoclonal antibodies, as well as antigen-binding fragments thereof. In embodiments of the invention, the tag comprising the detectable moiety is an aldehyde reactive probe comprising a detectable moiety (e.g., biotin or a fluorescent moiety), where the aldehyde reactive probe associates with AP sites.

Any suitable detectable moiety known in the art can be used in the practice of the present invention. In embodiments of the invention, the detectable moiety is a portion of the tag that associates with the damaged DNA, the reagent that indicates DNA replication and/or the reagent that associates with DNA. For example, the detectable moiety can be a portion of an antibody that associates with the damaged DNA or sites of DNA replication, which portion can be indirectly detected using another antibody directed against the first antibody (e.g. a rabbit anti-mouse antibody).

Alternatively, the detectable moiety can be an exogenous epitope or chemical label that is covalently attached to the tag or reagent that associates with the damaged DNA, the reagent that indicates DNA replication and/or a portion of the reagent that associates with DNA. The detectable moiety can be any exogenous label that can be detected using any method known in the art. According to this embodiment, the detectable moiety can be an epitope, an enzyme, a ligand, a receptor, an antibody or antibody fragment and the like. In representative embodiments, the detectable moiety is a hemagglutinin antigen, polyHis, biotin, Protein A, streptavidin, maltose binding protein, c-myc. FLAG, or an enzyme such as glutathione-S-transferase, alkaline phosphatase, horseradish peroxidase, β-glucuronidase, β-galactosidase or luciferase. Further, the detectable moiety can be, without limitation, a fluorescent moiety (e.g., Green Fluorescent Protein or a nanocrystal [e.g., a quantum dot such as a Qdot® Nanocrystal from Invitrogen]), a radioactive moiety and/or an electron-dense moiety such as a ferritin or gold particle(s).

The detectable moiety can be detected either directly or indirectly using any suitable method. For example, for direct detection, the tag or reagent can comprise a radioisotope (e.g., ³⁵S) and the presence of the radioisotope detected by autoradiography. As another example, the tag or reagent can comprise a fluorescent moiety and be detected by fluorescence as is known in the art. Alternatively, the tag or reagent comprising the detectable moiety can be indirectly detected, i.e., the detectable moiety requires additional reagents to render it detectable. Illustrative methods of indirect labeling include those utilizing chemiluminescence agents, chromogenic agents, enzymes that produce visible reaction products, and ligands (e.g., haptens, antibodies or antigens) that may be detected by binding to labeled specific binding partners (e.g., hapten binding to a labeled antibody or a first antibody binding to a second antibody).

In particular embodiments, the tag or reagent is an antibody or antibody fragment. A variety of protocols for detecting the presence of and/or measuring the amount of antibodies or other polypeptides are known in the art. Examples of such protocols include, but are not limited to, enzyme-linked immunosorbent assays (ELISA), radioimmunoassays (RIA), radioreceptor assay (RRA), competitive binding assays and immunofluorescence. These and other assays are described, among other places, in Hampton et al. (Serological Methods, a Laboratory Manual, APS Press, St Paul, Minn. (1990)) and Maddox et al. (J. Exp. Med. 158:1211-1216 (1993)). In a further embodiment, as described above for

In embodiments of the invention, detecting the tag or reagent comprising the detectable moiety comprises imaging the tag comprising the detectable moiety (e.g., by fluorescence microscopy). According to this embodiment, the image can be processed, e.g., using a computer-based method. For example, an algorithm can be used to determine the presence or amount of the detectable moiety above background or a threshold value.

Methods of detecting a detectable moiety are known in the art, for example, the detectable moiety can be a fluorescent moiety, which can be detected using fluorescence microscopy, which has the advantage that the DNA fiber can be simultaneously labeled with multiple tags, each comprising a different fluorescent moiety. Electron-dense moieties such as ferritin and gold particles can be detected using electron microscopy. The detection method can be a computer-based method.

In further representative embodiments, the detectable moiety is detected using a microfluidic or a nanofluidic device. For example, fluorescently labeled DNA damage and/or fluorescently stained DNA can be detected using a fluorescence detector in a microfluidic or a nanofluidic device. As an alternative, a detectable moiety (e.g., an antibody or other reagent) associated with a site of DNA damage can be used to increase the size of the DNA at the site of damage. The increase in size of the DNA can cause a change in the electrical current across the channel of a microfluidic or nanofluidic device as the DNA travels through the narrow channel. This change in current can be detected as a change in impedance or resistance across or along a microfluidic or nanofluidic channel, thereby providing a further method of detecting a detectable moiety and assessing DNA damage.

In embodiments of the invention, two or more forms (e.g., two, three, four or five forms) of DNA damage are detected simultaneously or sequentially in the same DNA fiber preparation. Thus, the method can comprise labeling the cellular DNA or DNA fiber with a second tag comprising a second detectable moiety that is different from the first detectable moiety (and, optionally, any other detectable moieties being used in the analysis to measure DNA replication, to measure total DNA, and the like), wherein the second tag associates with a different form of DNA damage than the first tag. Additional forms of DNA damage can be assessed in the same way, by using a tag that associates with the particular form of DNA damage and comprising a detectable moiety that differs from the other detectable moieties being used to measure other forms of DNA damage, DNA replication, total DNA, and the like.

In embodiments of the invention, clusters of DNA damage (e.g., AP clusters) are detected, e.g., regions of the DNA with a frequency of DNA damage sites that is greater than the average frequency.

The methods of the invention can be used to assess the amount and/or distribution of DNA damage. On addition, the distribution of DNA damage within specific regions of the genome can be assessed (e.g., in association with marker genes known to be linked to particular diseases and disorders). Marker genes include without limitation genes that are commonly mutated in a number of skin cancers and other cancers, for example, CDKN2A, p16 (Ink4a), p14 (ARF), p53, H-Ras, K-Ras, N-Ras, MYC, ABL, APC, BRCA1, BRCA2, SMH2, PTCH, RB, TP53, PTEN. Nrf2, and the like. Localization of DNA damage along the DNA fiber can be done using any method known in the art and as described herein, for example, fluorescent in situ hybridization (FISH) or hybridization with probes conjugated to electron-dense (e.g., ferritin or gold particles), radioactive moieties or any other detectable moiety (e.g., as described herein) and/or detection of changes in current across (transverse) or along the line of fluid flow (parallel) of a microfluidic or nanofluidic channel. Specific regions of the genome can also be localized by cutting the DNA with a restriction enzyme(s) (e.g., a rare cutter), measuring the length of the DNA fragment(s) and then, mapping that location(s) within the genome.

The DNA fibers can be prepared using any suitable method known in the art. In embodiments of the invention, the DNA fibers are formed from isolated DNA (e.g., the protein components of chromatin and other proteins associated with genomic DNA are substantially or completely removed, for example, at least about 50%, 60%, 70%, 80%, 90% or more are removed). In other embodiments, the DNA fiber is formed from chromatin (e.g., the protein components of chromatin including histones, enzymes, transcription factors, and the like are substantially retained, for example, at least about 50%, 60%, 70%, 80%, 90% or more is retained). The conditions under which the cell is lysed and/or the fibers are spread can be altered to affect whether protein components remain associated with the DNA fibers.

Methods of preparing DNA fibers or “DNA spreads” are known in the art. As one approach, the DNA fibers can be prepared on a microscope slide (e.g., a slide coated with silane [aminoalkylsilane]). Any other suitable support matrix can be used, e.g., a glass disc or a plastic slip (e.g., that is optically inert). In one illustrative, but nonlimiting, embodiment of making DNA fibers from isolated DNA the cell suspension is applied to the slide and allowed to evaporate until almost, but not completely, dried and then overlaid with a buffer comprising an anionic surfactant such as SDS (e.g., 0.5% SDS). The slide can then be tilted to allow the cell lysate to slowly move down the slide, and the resulting DNA spreads can be air-dried, fixed in 3:1 methanol/acetic acid, air-dried, and then stored frozen. In embodiments of the invention, the sample is maintained at an angle from about 10, 12 or 15 to about 20, 21, 22, 23, 24, 25, 26, 27, 28, 29, 30, 32, 35, 38 or 40 degrees from horizontal or from about 15 or 20 to about 25, 30 or 35 degrees from horizontal during the lysing/spreading process. In embodiments of the invention, the sample is maintained at an angle of about 20, 21, 22, 23, 24 or 25 degrees from horizontal during the lysing/spreading process. In general, the angle is selected so that the fibers are stretched enough so that they and do not overlap each other, but are not stretched so much that there is an undue amount of fragmentation. Further, when making DNA fibers from isolated DNA, the distribution of the cells can be modified so that the individual fibers are visible and overlap is reduced or minimized. As a nonlimiting illustration, when using a slide to prepare the DNA fibers, two microliters of solution containing about 2 to 400 cells per microliter can be used (for a total of about 4 to 800 cells per slide).

To prepare chromatin fibers, the lysis/spreading buffer can be modified from the buffer used to produce fibers of isolated DNA in order to retain the protein components. An exemplary lysis/spread buffer comprises a nonionic surfactant and a protein denaturing agent (e.g., 1% Triton X-100 and 0.2 M urea). When the DNA fiber comprises chromatin, in representative embodiments, the sample is maintained in an essentially horizontal orientation during the lysing/spreading process (e.g., less than about 10, 5, 3, 2 or 1 degrees from horizontal). Without wishing to be limited by any particular theory of the invention, it is advantageous to allow the recession of the meniscus to pull (stretch) the chromatin fibers, rather than using the angle of the slide. As a nonlimiting example, when using a slide to prepare the chromatin fibers, from about 2000, 4000 or 8000 to about 12,000, 15,000 or 20,000 cells can be used to prepare the chromatin fibers.

Quite surprisingly, the inventors have found that a lysis/spreading buffer that contains reagents such as Triton X-100 and urea, which would be expected to extract the proteins, can be used to prepare chromatin fibers. Without being limited by any theory of the invention, one can optimize the recession of the meniscus (e.g., due to evaporation) to optimize the number of cells that will be suitable for analysis of DNA damage, DNA repair proteins, checkpoint proteins, DNA replication proteins, and the like. As a non-limiting example, when drying cells on slides, allowing the meniscus to retract from about the outer one-third of the cover slip works well for such evaluation. The cells in the center of the cover slip, which have been in contact with the buffer for a longer period of time will often appear as a “halo,” with the DNA standing out from the nucleus. To avoid this problem, a very short recession time for the meniscus can be used; however, those skilled in the art will appreciate that fewer cells will be produced that are suitable for analysis (only the cells along the outermost edge where the meniscus has receded). In embodiments of the invention, the meniscus is allowed to recede for less than about 24 hours, less than about 18 hours, less than about 12 hours, less than about 10 hours, less than about 8 hours, less than about 6 hours, less than about 5 hours, less than about 4 hours, less than about 3 hours, less than about 2 hours or less than about 1 hour. In embodiments of the invention, the meniscus is allowed to retract for about 0.5, 1, 2, 3 or 4 hours to about 8, 10, 12 or 15 hours. The amount of Triton X-100 and/or urea can be modified if shorter or longer drying times are used to achieve the desired level of usable chromatin fibers.

Microfluidics can also be used to form DNA fibers. As one approach, cells can be lysed to prepare DNA fibers from isolated DNA or from chromatin (e.g., as described herein), the DNA is purified and both damaged and DNA can be labeled (e.g., fluorescently labeled through a DNA intercalator or an antibody that detects DNA that is fluorescently labeled). After labeling the DNA and DNA damage, an aliquot of the sample is placed into a microfluidics or nanofluidics chamber that straightens the DNA. The straightened DNA can then be passed through a detector (e.g., a fluorescence detector). In representative embodiments, the detector measures the amount of time a certain signal (e.g., the one for total DNA) lasts and how many times that signal is interrupted by a different signal (e.g., the signal associated with the damaged DNA). Methods of using microfluidics to prepare DNA fibers formed from isolated DNA have been described, see, e.g., U.S. Pat. No. 6,544,734. In alternative embodiments, at least one pair of electrodes (transverse to or parallel with the channel of the chamber) can be used to assess (detect) DNA damage. In this latter case, the DNA may or may not be labeled. In representative embodiments, as the DNA moves through the channel, changes in the electrical current that result from changes in the DNA associated with damage to the DNA can be detected, thereby assessing damage to the DNA.

As another approach, whole cells can be injected into the microfluidic device, and the cells are placed into a buffer (e.g., a hypotonic buffer) that separates the nucleus from the rest of the cell. In representative embodiments, the nucleus is then lysed. In the case of DNA fibers formed from isolated DNA, the lysis buffer essentially removes any proteins that are associated with the DNA. DNA (i.e., total DNA) can then be stained and damaged DNA labeled (e.g., fluorescently labeled through a DNA intercalator or an antibody that detects DNA that is fluorescently labeled). Afterwards, the DNA undergoes can optionally undergo a number of wash steps to remove unbound DNA dye and label. Finally, the DNA is passed through a detector (e.g., a fluorescence detector) or at least one pair of electrodes to detect total and damaged DNA.

Chromatin fibers can be advantageously used to study proteins associated with DNA repair processes, for example, to evaluate whether some of these pathways are impaired and/or under-utilized in certain disease states such as cancer. According to this embodiment, the invention can comprise detecting the abundance and/or localization of particular proteins (e.g., Ogg1, BRCA1, BRCA2, Chk1, PARP1) co-localized with damaged regions of the DNA.

Chromatin fibers are also useful for detecting checkpoint proteins, which are associated with the cell cycle. The presence of different checkpoint proteins can be an indication that different phases of the cell cycle have been affected by DNA damage. For example, if cells appear to be trapped in S phase, that can be an indication that DNA replication has been adversely affected by the DNA damaging agent(s). Such cells may be at an increased risk for genomic instability and disease formation.

As another option, the functionality of cellular DNA repair processes can be used to assess a subject's risk for developing a disease or disorder correlated with DNA damage such as cancer. For example, cells (e.g., lymphocytes) can be removed from a subject, exposed to one or more DNA damaging agents, and the degree of DNA damage before and after such exposure can be determined. Subjects that have a reduced ability (e.g., as compared with a reference population) to repair DNA following exposure to the DNA damaging agent(s) may be at an elevated risk of developing a disease such as cancer that is linked with DNA damage.

The methods of the invention can further comprise identifying or assessing an area of DNA replication along the DNA fiber. Optionally, DNA damage in the area of DNA replication is assessed. Methods of identifying areas of DNA replication are described herein e.g., using nucleotide precursors comprising a detectable moiety and/or detecting a DNA replication protein(s)). Accordingly, in embodiments of the invention, the method comprises labeling the DNA fiber with a reagent comprising a detectable moiety that indicates (e.g., assesses) DNA replication. In representative embodiments, the reagent that indicates DNA replication is a nucleotide precursor comprising a detectable moiety end/ore reagent (e.g., an antibody) comprising a detectable moiety that recognizes a replication and/or checkpoint protein.

Different types of damage are associated with different repair pathways; thus, the presence of different repair proteins can be used as an indirect method of identifying the underlying type of DNA damage. Thus, the invention also encompasses methods of assessing the type and/or distribution of DNA damage in a cell (e.g., a cell from a subject) to determine the type of DNA damaging agent(s) to which the cell or subject has been exposed. For example, 8-oxoguanine glycosylase (OGG1) is involved in the repair of 8-oxo-dG, which is caused by oxidative stress. ATRIP and phospho-RPA are markers for stalled replication forks, which are often seen after UV damage, and 53bp1 is a marker for double strand breaks, which are routinely seen following ionizing radiation and also very high doses of UV.

Further, in representative embodiments, the presence of DNA repair proteins in chromatin fibers is used as a marker of DNA damage.

The invention can be practiced as a qualitative, semi-quantitative, or quantitative method. For example, qualitative methods can be used to detect the presence or absence of DNA damage. Semi-quantitative methods can be used to determine whether the level of DNA damage rises above a threshold value (e.g., a value associated with increased risk of disease or otherwise considered unsafe) and/or to score damage by general categories such as “slight,” “moderate,” and “severe.” Quantitative methods can be used to determine a relative or absolute amount of DNA damage.

A threshold or cutoff value can be determined by any means known in the art, and is optionally a predetermined value. In particular embodiments, the threshold value is predetermined in the sense that it is fixed, for example, based on previous determinations of the level of DNA damage associated with increased risk of disease or disorder or otherwise deemed unsafe. Alternatively, the term “predetermined” value can also indicate that the method of arriving at the threshold is predetermined or fixed even if the particular value varies depending on the methodology used or may even be determined for every set of samples evaluated.

In quantitative methods, the amount of DNA damage (e.g., number of damage sites) can optionally be standardized, e.g., to the amount of damage per cell, per chromosome or per unit of nucleotides (e.g., 10⁶ or 10⁹ nucleotides). Reagents are known in the art for detecting and, optionally, measuring DNA (i.e., total DNA). Advantageously, the reagent can comprise a detectable moiety that differs from the detectable moiet(ies) being used to label the DNA damage so that total and damaged DNA can be measured simultaneously. Methods of visualizing and/or measuring DNA are known in the art; for example, using a DNA stain or dye such as YOYO-1 (Invitrogen), DAPI (4′,6-diamidino-2-phenylindole) or a Hoechst stain (e.g., Hoechst 33258 or Hoechst 33342), which are fluorescent stains that bind strongly to DNA.

In embodiments of the invention, a correlation is established between the type, amount and/or distribution of DNA damage and the increased risk of developing a disease or disorder (e.g., any disease or disorder as described herein, such as cancer). A population of subjects can be evaluated to establish the correlation, and then a test subject can be evaluated for the type, amount and/or distribution of cellular DNA damage, and these results used to predict whether the subject has an the increased risk of developing the disease or disorder as a result of the type, amount and/or distribution of DNA damage present. Such methods can be qualitative, semi-quantitative or quantitative.

In particular embodiments, the number of AP clusters (or clusters of other forms of DNA damage) are counted per cell or per region of the genome and used to assess risk of a disease or disorder (e.g., cancer) and/or to evaluate the safety and/or protective effects of an agent and/or to assess prior exposure to DNA damaging agents.

The methods of the invention can also be manual, semi-automated, or completely automated, for example, semi-automated or automated with a machine. As one non-limiting illustration, the staining of slides to visualize the tag comprising the detectable moiety can be automated and/or the detection of changes in an electrical current across or along a micro- or nanofluidic channel resulting from damage in the DNA (with or without a tag) can be automated.

The term “antibody” or “antibodies” as used herein refers to all types of immunoglobulins, including IgG, IgM, IgA. IgD, and IgE. The antibody can be monoclonal or polyclonal and can be of any species of origin, including (for example) mouse, rat, rabbit, horse, goat, sheep, camel, or human, or can be a chimeric antibody. See, e.g., Walker et al., Molec. Immunol. 26:403 (1989), The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980.

Antibody fragments included within the scope of the present invention include, for example, Fab, Fab′, F(ab′)₂, and Fv fragments; domain antibodies, diabodies; vaccibodies, linear antibodies; single-chain antibody molecules; and multispecific antibodies formed from antibody fragments. Such fragments can be produced by known techniques. For example, F(ab′)₂ fragments can be produced by pepsin digestion of the antibody molecule, and Fab fragments can be generated by reducing the disulfide bridges of the F(ab′)₂ fragments. Alternatively. Fab expression libraries can be constructed to allow rapid and easy identification of monoclonal Fab fragments with the desired specificity (Huse et al., Science 254:1275 (1989)).

Antibodies may be altered or mutated for compatibility with species other than the species in which the antibody was produced. For example, antibodies may be humanized or camelized. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins recipient antibody) in which residues from a complementarity determining region (CDR) of the recipient are replaced by residues from a CDR of a non-human species (donor antibody) such as mouse, rat or rabbit having the desired specificity, affinity and capacity. In some instances. Fv framework residues of the human immunoglobulin are replaced by corresponding non-human residues, Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework (FR) regions (i.e., the sequences between the CDR regions) are those of a human immunoglobulin consensus sequence. The humanized antibody optimally also will comprise at least a portion of an immunoglobulin constant region (Fc), typically that of a human immunoglobulin (Jones et al., Nature 321:522 (1986); Riechmann et al., Nature, 332:323 (1988); and Presta, Curr. Op. Struct. Biol. 2:593 (1992)).

Having described the present invention, the same will be explained in greater detail in the following examples, which are included herein for illustration purposes only, and which are not intended to be limiting to the invention.

### Example 1

### Materials and Methods

### Cell Lines and Culture Condition

Avian DT40 cells (38, 39) were grown at 39.5° C., the normal Avian body temperature, in a humidified atmosphere supplemented with 5% CO₂ as a suspension in RPMI-1640 (Invitrogen) supplemented with 10% fetal bovine serum (Sigma), 1% chicken serum (Sigma and Invitrogen), and containing 100 μg/ml penicillin and 100 μg/ml streptomycin (Invitrogen).

### Detection of Endogenous AP Sites by Slot-Blot Analysis

DNA was isolated from DT40 cells in normal culture conditions or DT40 cells experiencing oxidative stress and processed for slot-blot analysis as described previously (2).

### DNA Labeling and Fiber Spreading

The detection of areas undergoing replication in isolated DNA fibers was originally performed by Bensimon (40, 41) and later modified by Jackson and Pombo (42) to generate DNA fibers directly from lysed cells instead of using purified DNA. The DNA fiber extension methodology used in this paper is a modified version of the protocol initially described by Merrick et al. (43), which is a modification of Jackson and Pombo's method. Briefly, cells growing in culture were first labeled for 10 min in medium with 100 μM iododeoxyurine (IdU), and then centrifuged to remove the medium containing IdU. Cells were resuspended in unlabeled medium and exposed to H₂O₂ for 10 min. H₂O₂ exposure was terminated by the addition of catalase (3 U/mL) for 10 min then centrifuged, and thereafter the cells were resuspended in medium with 50 μM chlorodeoxyuridine (CIdU) for 20 min to provide a second DNA label. After exposure to the second halogenated nucleotide, the cells were harvested by centrifugation and resuspended in ice cold PBS at about 200 cells/μl.

For the preparation of the DNA fiber spreads upon slides, two μl of cell suspension were spread on a SILANEPREP™ slide (Sigma-Aldrich, S4651), close and parallel to the label. The sample was allowed to evaporate until almost, but not completely dry and then overlaid with 10 μl of spreading buffer (0.5% SDS in 200 mM Tris-HCl (pH 7.4), 50 mM EDTA). After ˜10 min the slide was tilted at ˜20° to 40° from horizontal to allow the cell lysate to slowly move down the slide, and the resulting DNA spreads were air-dried, fixed in 3:1 methanol/acetic acid for 2 min, air-dried overnight, then stored at −20° C. for at least 24 h.

For the detection of IdU and CIdU within the DNA fiber spreads, the slides were treated with 2.5 M HCl for 30 min, washed several times in PBS, and blocked in 3% bovine serum albumin in PBS for 60 min. The slides were incubated at room temperature with the antibodies indicated below, rinsed three times in PBS, and incubated for 30 min in blocking buffer between each of the following incubations: 1) 1 hr in 1:500 rat anti-bromodeoxyuridine (detects CIdU) (OBT0030, Accurate) plus 1:500 mouse anti-bromodeoxyuridine (detects IdU) (Becton and Dickinson); 2) 30 min in 1:500 Alexafluor 488-conjugated chicken anti-rat (Molecular Probes) plus 1:500 Alexafluor 594-conjugated rabbit anti-mouse; and 3) 30 min in 1:500 Alexafluor 488-conjugated goat anti-chicken plus Alexafluor 594-conjugated goat anti-rabbit. In addition, prior to the blocking step between the first and second antibody incubations, the slides were placed for 15 min in a stringency buffer containing 10 mM Tris HCl (pH 7.4), 400 mM NaCl, 0.2% Tween-20, 0.2% Nonidet P40 (NP40) to remove any nonspecifically bound primary antibodies. The slides were rinsed three times in PBS and mounted in antifade (UNC Microscopy Core). Microscopy was carried out using an Olympus FV500 confocal microscope in sequential scanning mode,

### Fluorescence Visualization of AP Sites

We tried three approaches for the labeling and visualization of AP sites in the DNA fiber spreads. Approach 1: biotin-tagged aldehyde reactive probe (ARP), which reacts with the ring-open form of AP sites to generate a biotin-tagged AP site, was added to the cells one hour before DNA fibers were prepared. Approach 2: DNA fibers were prepared first, and then the AP sites were reactively tagged with ARP. Approach 3: same as Approach 2, but using a fluorescent form of ARP called F-ARP. Using confocal microscopy, either the fluorescently tagged AP sites in the fibers could be directly visualized, or the biotin-tagged AP sites could be detected using fluorescent antibodies against biotin, DNA was labeled with a DNA dye (YOYO-1. Invitrogen) which provides a bright green signal when it associates with DNA. Regardless of the method used, we found that the distribution of AP sites in the DNA fiber spreads was equivalent. Since all three approaches seemed to give similar results and Approach 2 required the least time and cost for reagents, we chose to use that methodology for our analyses.

### Image Processing and Calculation

Images were processed using ImageJ software (Abramoff et al., “Image Processing with Image J”. Biophotonics international 11(7): 36-42, (2004)). To determine the number of AP sites within a given field of DNA fibers, the average fluorescence intensity per nucleotide (nt) was determined as follows: 1) a section of one of the fibers was erased and the total intensity of the image was recalculated; 2) the intensity of the erased fiber was determined by subtracting the new total fluorescent intensity from the previous total intensity; 3) the number of nt in the erased DNA fiber was determined by measuring the erased fiber length in microns and then multiplying that value by 6000 nt per micron (i.e., 3000 bp/micron×2) (44); 4) finally, the average fluorescent intensity per nt was obtained by dividing the fluorescent intensity of the DNA fiber by its length expressed in nt. For each image, we determined the fluorescent intensity of at least 5 different fibers located in different areas of the image. This allowed on to calculate the average fluorescence intensity per nt for that image. To obtain the total amount of DNA (expressed in nucleotides) in a given image, the total fluorescence intensity of the image was divided by the average intensity per nt for that image.
The intensity of the Fiber, F, equals the total green fluorescence of the image before the fiber was subtracted minus the intensity after the fiber was subtracted, I_{b} and Iₐ, respectively.

F=I_{b}−Iₐ
The average fluorescent intensity of each nucleotide

$$\overline{nt\_int} = \frac{1}{n} {\sum }_{i = 1}^{n} \frac{{F}_{i}}{6000 {L}_{i}} = \frac{1}{n} ( \frac{{F}_{1}}{6000 {L}_{1}} + … + \frac{{F}_{n}}{6000 {L}_{n}} )$$

where n equals 5 (the number of fibers measured per image), intensity of the fiber ═F, 6000 is the number of nt per micron of DNA and length of fiber=L. The total number of nucleotides in the image, T_{nucleotides}, equals

$${T}_{nucleotides} = \frac{{I}_{b}}{\overline{nt\_int}}$$

We also devised a method to determine the total number of AP sites (labeled by red fluorescence) on well-defined DNA fibers in the same images. We used the ImageJ program to subtract background red fluorescence and focus the quantitative analysis on AP sites that met empirically determined criteria, We examined an image and compiled a distribution of the sizes and intensities of the entire red fluorescent signal. We determined empirically that a true single AP site had an area of 1 (as defined by the ImageJ software) and intensity between 45 to 80 intensity units (3). Since we were interested only in red signal associated with AP sites overlapping with clearly identifiable green DNA fibers, we used the co-localization function of ImageJ to identify red signal that co-localized with green DNA fibers. To exclude from analysis any red signal that was not associated with AP sites (i.e., red signal below the intensity of 45), we evaluated different settings for the lower limit threshold for red fluorescence, We found that the red fluorescence signal decreased as the threshold was increased, up to 50 intensity units. Subsequent small increases of the threshold did not reduce the number of apparent AP sites detected and only slightly reduced the overall red fluorescence signal. Based on these observations, we set the lower limit threshold for red signal at 50 (anything above 50 was determined to be an AP site signal and anything below was not). The red signals that co-localized with green DNA fibers were counted using the particle counter function of ImageJ, Red signal with an area equivalent to twice the signal of one AP site was counted as 2 AP sites, three times the signal as 3 AP sites, etc.

To determine the number of nt that had been replicated (i.e., incorporated IdU or CIdU) in normal culture conditions, the total amount of fluorescence from both IdU and CIdU was measured. The number of AP sites co-localized in areas where DNA had replicated was assessed using the co-localization function provided by Image J. In this series of experiments, AP sites were marked by blue signal and those that co-localized with areas undergoing replication were counted using the particle counter function of Image J. To determine the amount of replicating DNA expressed in nt, the total amount of red and green fluorescence (IdU and CIdU, respectively) was determined and then divided by fluorescence intensity per nt, as described above.

The intensity of Red Track, R, equals the total red fluorescence intensity of the whole image before the fiber was subtracted minus the intensity after the fiber was subtracted, T_{rb} and Tᵣₐ, respectively.

R=T_{rb}−Tᵣₐ

The average fluorescent intensity of each nucleotide

$$\overline{r\_nt \_int} = \frac{1}{n} {\sum }_{i = 1}^{n} \frac{{R}_{i}}{6000 {L}_{i}} = \frac{1}{n} ( \frac{{R}_{1}}{6000 {L}_{1}} + … + \frac{{R}_{n}}{6000 {L}_{n}} )$$

where n equals 5 (the number of fibers measured per image), intensity of the red track=R, 6000 is the number of nt per micron of DNA and length of fiber=L. The total number of red nucleotides in the image, T_{red}_{_{—}}_{nucleotides}, equals

$${T}_{red\_nucleotides} = \frac{{T}_{rb}}{\overline{r\_nt \_int}}$$

Intensity of Green Track, G, equals the total green fluorescence of the image before the fiber was subtracted minus the intensity after the fiber was subtracted, T_{gb} and T_{ga}, respectively.

G=T_{gb}−T_{ga}

The average fluorescent intensity of each nucleotide

$$\overline{g\_nt \_int} = \frac{1}{n} {\sum }_{i = 1}^{n} \frac{{G}_{i}}{6000 {L}_{i}} = \frac{1}{n} ( \frac{{G}_{1}}{6000 {L}_{1}} + … + \frac{{G}_{n}}{6000 {L}_{n}} )$$

Where n equals 5 (the number of fibers measured per image), intensity of the green track=G, 6000 is the number of nt per micron of DNA and length of fiber=L. The total number of green nucleotides in the image, T_{green}_{_{—}}_{nucleotides}, equals

$${T}_{green\_nucleotides} = \frac{{T}_{gb}}{\overline{g\_nt \_int}}$$

### Statistical Analysis.

To estimate the effect of H₂O₂ on AP site formation in DNA fibers globally and areas undergoing replication, a Poisson regression was used to model the distribution of AP sites. A Wald test was used to determine the statistical significance of the H₂O₂ effect. All statistical analyses were done using SAS 9.2 (SAS Institute Inc., Cary, N.C.).

### Example 2

### Results and Discussion

### Average Number of AP Sites in DNA Fiber Spreads

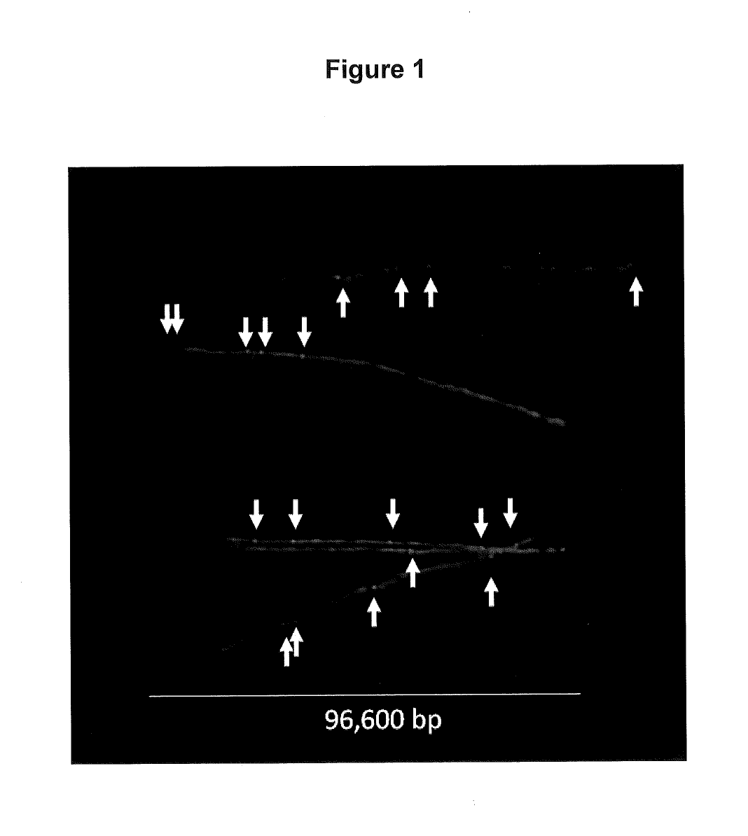

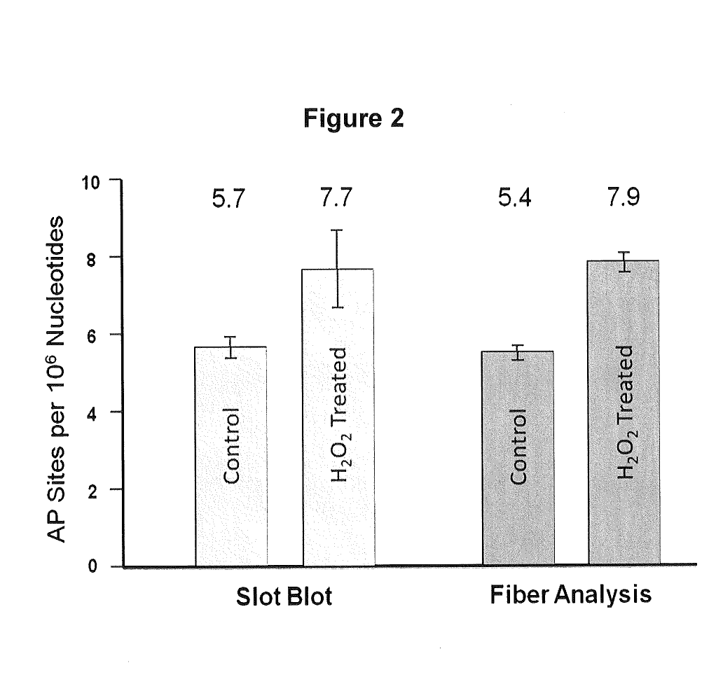

To quantify the number of AP sites per 10⁶ nt in the YOYO-labeled DNA fiber spreads, we determined the total amount of DNA (expressed in nucleotides, nt) in a given image by dividing the total green fluorescence intensity of the image by the average intensity per nt for that image, as outlined in Material and Methods (Example 1). We then determined the total number of AP sites located in well-defined DNA fibers in the image by counting the number of AP sites that co-localized to those fibers (see Material and Methods Section; Example 1) FIG. 1 presents a composite image of a number of isolated DNA fibers that were observed in our samples. Using this approach, we analyzed over 10⁹ nt of DNA and determined that our sample of DNA from cells growing in normal culture conditions contained a basal value of 5.4 AP sites per 10⁶ nt, while slot blot analysis gave a value of 5.7 AP sites per 10⁶ nt (FIG. 2). The number of AP sites detected in DT40 cells in this study is similar to what was found previously in HeLa cells and calf thymus DNA using a different methodology (2). When we analyzed 7×10⁸ nt in DNA fiber spreads from cells that were exposed to 20 μM H₂O₂, we found that the number of AP sites increased to 7.9 per 10⁶ nt, also consistent with the 7.7 AP sites per 10⁶ nt found by slot blot analysis (FIG. 2). The observed difference in AP sites in the absence and presence of H₂O₂ (5.4 AP sites per 10⁶ nt versus 7.9 AP sites per 10⁶ nt, respectively) is statistically significant with p<0.0001, as determined using the Poisson regression model. It was interesting to note that there was considerably less variance in the values of AP sites per 10⁶ nt obtained using fiber analysis than there was using the slot blot technique. Nonetheless, in view of the far greater effort involved in making these measurements of AP sites per 10⁶ nt by fiber analysis, the slot blot technique remains the logical choice for routine assessment of the quantity of AP sites,

### AP Sites in Areas of DNA Undergoing Replication

In this study we observed that many AP sites occurred in clusters in both untreated and H₂O₂-treated cells, confirming the observations previously made in HeLa cells and calf thymus DNA. Furthermore, clustering appeared to be more common in the H₂O₂-treated cells (FIG. 1). As noted earner, we hypothesized that AP site formation in DNA might result from a higher propensity for ROS to attack chromatin that has an unusually open or exposed state, such as is found in genomic regions undergoing transcription or DNA replication. Recently, others and we have reported the capability to identify where DNA replication is occurring in extended DNA fibers (42, 43, 45). By incorporating two thymidine analogs in short sequential pulses, the direction of DNA replication can be determined and replication structures such as origins and termination site can be identified (42-46). These new techniques for analysis of DNA replication, when combined with our demonstration of AP sites using fluorescent probes, allow us to examine the formation of AP sites with regard to areas of DNA replication and to address very important questions about how replication is affected by oxidative stress.

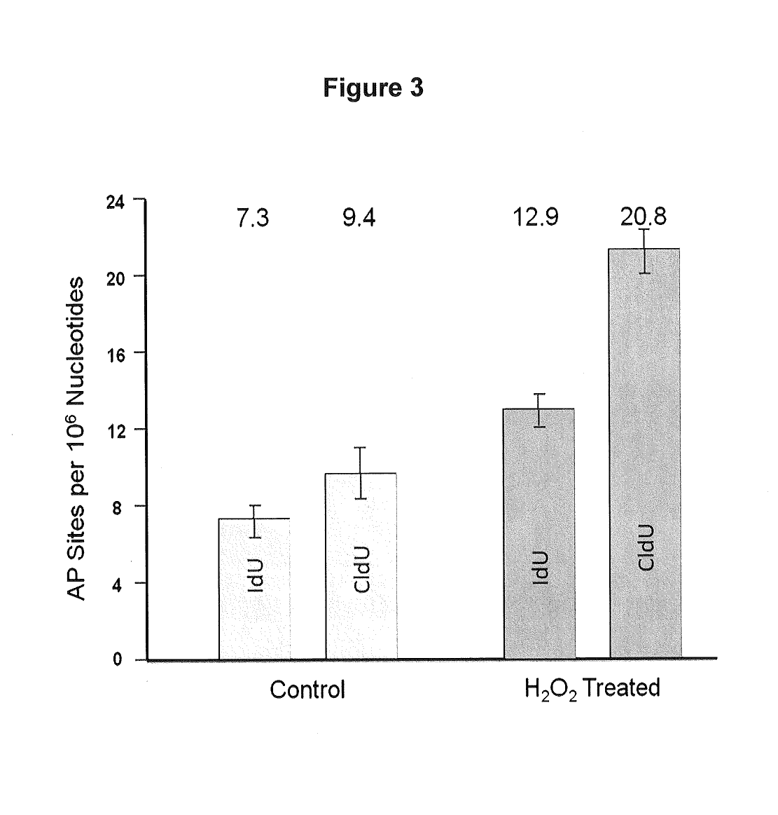

To determine the number of AP sites per unit length of DNA in areas undergoing replication, cells were first pulsed with IdU (a nucleotide precursor), and then pulsed with CIdU (a different precursor). To determine the effects of oxidative stress, cells were exposed to H₂O₂ between the two pulses, while control cells were treated similarly but without inclusion of H₂O₂. Fluorescently labeled AP sites were readily detected and could be quantified with respect to the fluorescent tracks of IdU and CIdU labeled DNA (see Materials and Methods). In the control cells not exposed to H₂O₂, we found that the number of AP sites in areas that were labeled with IdU was 7.3 AP sites per 10⁶ nt while in areas incorporating CIdU that replicated later the number was 9.4 AP sites per 10⁶ nt (FIG. 3). Both of these values were higher than the overall amount of AP site formation throughout the genome, which was found to be 5.4 AP sites per 10⁶ nt (FIG. 2); these differences are statistically significant with p<0.0001, as determined by the Poisson regression model. This result indicates that AP sites are 1.5- to 2-fold (50 to 100%) more likely to be present in areas where DNA replication is in progress. It does not, however, distinguish whether more AP sites are formed in these regions or whether they accumulate there because they are not removed as efficiently from these areas as from other genomic sites.

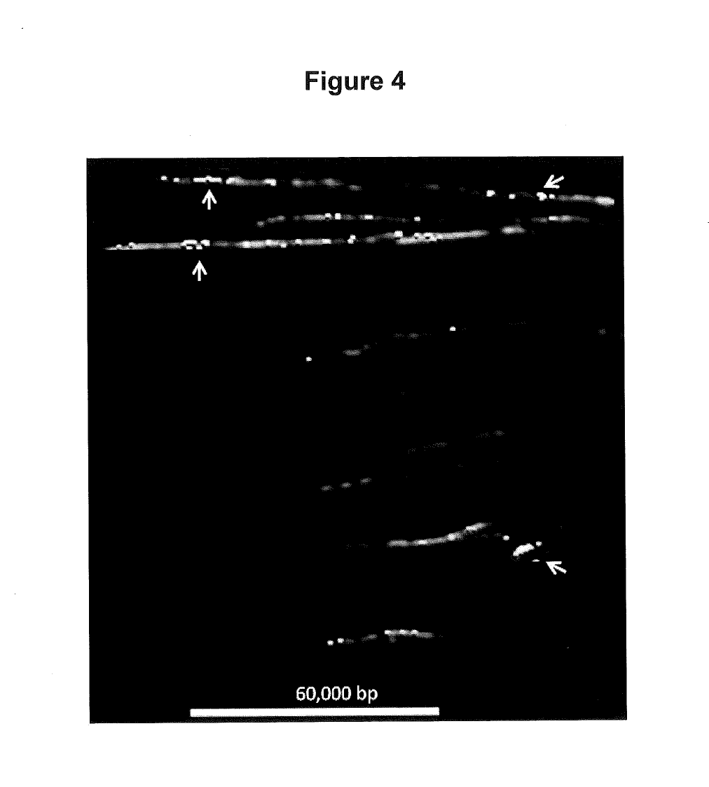

Finally we examined AP site formation in regions undergoing DNA replication in cells that had been further stressed by exposure to H₂O₂. Exposure to H₂O₂ occurred during the interval between the first pulse labeling of replicating DNA with IdU and the start of the second pulse labeling of replicating DNA with CIdU. While the number of AP sites per 10⁶ nt found globally in DNA exposed to H₂O₂ was 7.9, in the IdU tracks this increased to 12.9 and in the CIdU tracks it increased to 20.8. Similar to the results shown for AP sites in replicating regions where H₂O₂ was not added, AP sites per 10⁶ nt in replicating regions exposed to H₂O₂ are increased by 50 to 150%. These differences in AP sites per 10⁶ nt observed at replication sites are statistically significant with p<0.0001, as determined by the Poisson regression model. The greater increase in the formation of AP sites in areas undergoing replication indicates sites of replication are particularly vulnerable to the formation of AP sites by ROS-induced oxidative stress. AP sites were distributed as single and multiple events in the areas undergoing replication. Clustering of AP sites was detected in areas of the genome undergoing replication (FIG. 4), particularly in areas of transition between red and green label, that is, in areas replicated during exposure to hydrogen peroxide (FIG. 4). However, not all transitional areas had clusters. Occasionally we detected small stretches with only green label (CIdU, second pulse) in which AP sites were clustered heavily (one example is illustrated in FIG. 4). These areas represent origins of replication that were activated during or after exposure to hydrogen peroxide and began replicating in the presence of the second (green) pulse. We interpret this observation as indicating that some replication origins are extraordinarily sensitive to the effects of oxidative stress (AP site formation). The observation that there is an increased density of AP sites in regions of DNA fibers replicated during or after exposure to H₂O₂ suggests that open regions in the chromatin that form at or ahead of active replication forks are preferential targets for oxidative damage. These results are consistent with earlier observations that regions of DNA that were replicated while they were exposed to benzo(a)pyrene-diol-epoxide were more extensively adducted than nearby unreplicated regions near the replication fork (47).

We applied our ability to detect AP sites in replicating DNA to determine also whether replication forks prematurely terminate when they reach AP sites, or they are able to bypass the damage and continue to replicate the DNA template. As shown in FIG. 4, AP sites can clearly be detected in the CIdU tracks. Thus, it appears that replication forks are able to advance past these lesions even though they were not yet repaired, and the process proceeds rapidly since we can see multiple AP sites in tracks generated by a 20 min labeling with CIdU.

AP site clustering was once thought to occur only as a result of ionizing radiation (48-50). However, recent research suggests that clustering may be a normal occurrence within cells (34-36), most likely due to endogenous ROS, and may be more prevalent in tumors (33). The occurrence of these clusters in the genome of normal cells leads us to believe that there may be regions within the genome with increased vulnerability to ROS damage, such as regions undergoing replication. Our current analysis supports this assertion, as DNA that is in the process of replicating acquires 50 to 150% more AP sites than DNA that is not replicating, or replicated just prior to H₂O₂ exposure, even though the type of oxidative damage induced in areas undergoing replication is similar to what is found in bulk DNA (i.e., there are regions without any damage, regions with a single AP site and those that contain many AP sites (clusters)). We also detected clusters of AP sites in some regions between adjoined IdU and CIdU tracks (FIG. 4A, top of figure), and also in newly initiated origins, indicating that some areas of the genome undergoing replication may constitute preferential targets for AP site formation and cluster formation. An uneven distribution of AP sites (La, clustering) may imply that the detrimental effects of ROS in the development of disease may not simply be due to the total number of AP sites present, but to how AP sites are distributed in the genome during replication.

The new technology presented here makes it possible to analyze a large number of genomic DNA regions during metabolically important stages, such as replication (as shown in this paper) and transcription. This technology can be applied to detecting virtually every type of DNA damage. We demonstrated that replicating DNA is more vulnerable to the attack of ROS, as shown here by the increased level of AP site formation in regions labeled during DNA replication. This analysis can be performed with even more specificity by determining the genomic location of sites of replication that show enhanced vulnerability. This can be accomplished, for example, by coupling the methods described herein for detection of DNA damage in DNA fibers with fluorescent in situ hybridization (FISH) to localize the damage sites in selected genomic regions that are identified by hybridization of fluorescent genomic probes. The use of hybridization probes coupled to other detectable moieties can also be used to localize specific genomic regions.

### Example 3

### Detection of Multiple Types of DNA Damage in DNA Fibers

DNA fibers can be used to assess two or more types of DNA damage. Logarithmically growing human normal human fibroblasts were pulsed with iododeoxyuridine (IdU), exposed to 50 μM hydrogen peroxide, and then pulsed with chlorodeoxyuridine (CIdU). Approximately 400 cells were applied to a glass slide and treated with lysis buffer as described in Example 1 after which the slides were placed at an angle (about 30 degrees from horizontal) causing the DNA fibers to flow out (combing). The slides were then fixed. Immunostaining with fluorescent antibodies (AlexaFlour 594 for IdU and Alexaflour 488 for CIdU) was then performed to detect the presence of the halogenated uracil with red staining indicating regions with incorporated IdU and green staining regions with incorporated CIdU. DNA damage was detected by first using a chemical reagent that tags the damage with biotin and then that tag is immunostained with fluorescent antibodies (AlexaFlour 647) against the tag. The replication tracks and DNA damage were then observed on a confocal microscope.

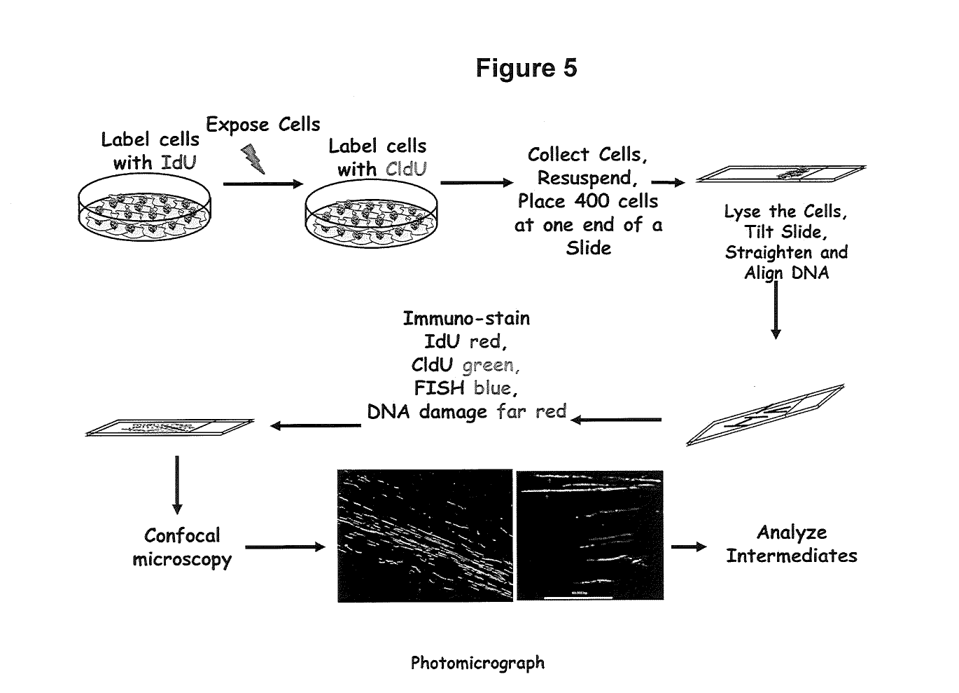

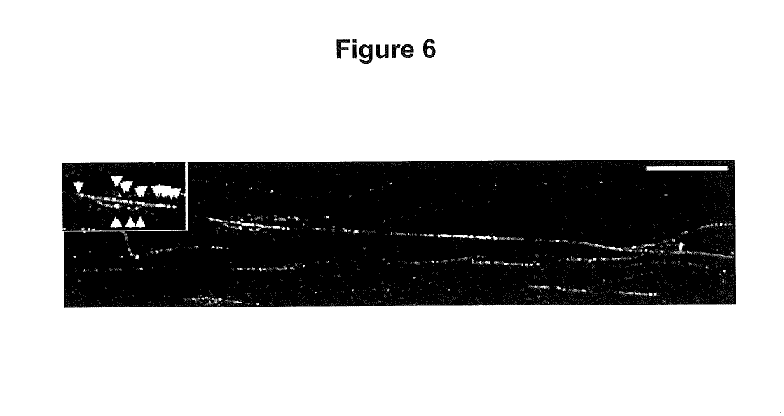

A schematic of this protocol is shown in FIG. 5. The results are shown in FIG. 6. CIdU (green) and CPDs (red) were detected in DNA fiber spreads generated from cells that were irradiated with 1 J/m2 UVC. The inset in FIG. 6 shows areas undergoing replication and CPD sites at higher magnification.

To do the above-mentioned detection, cells were exposed to UVC and subsequent CIdU incorporation was performed and detected as described above. CPDs were visualized using commercially available antibody. Using these methods, we can simultaneously detect CPDs and areas undergoing replication. The binding of anti-CPD antibodies does not interfere with the detection of CIdU tracts. This technique is able to identify regions of the DNA fibers with a single photolesion, as well as regions with multiple CPDs in close proximity.

The spacing of the DNA fibers can be adjusted by altering the concentration of the cells being lysed, such that the majority of the fibers are not overlapping.

### Example 4

### Detection of Stalled Replication Forks and Double Strand Breaks with Chromatin Fibers

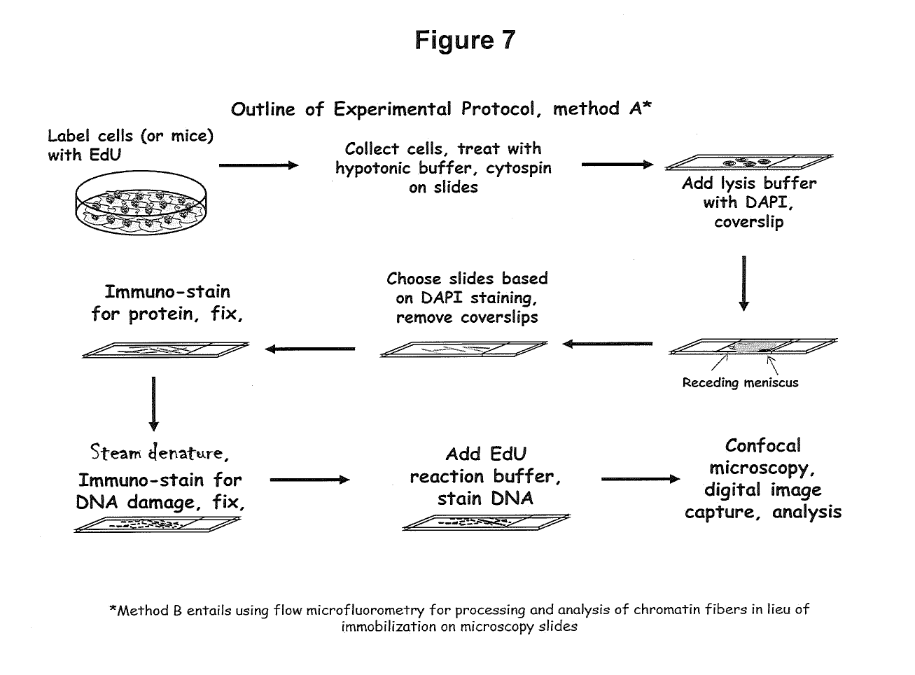

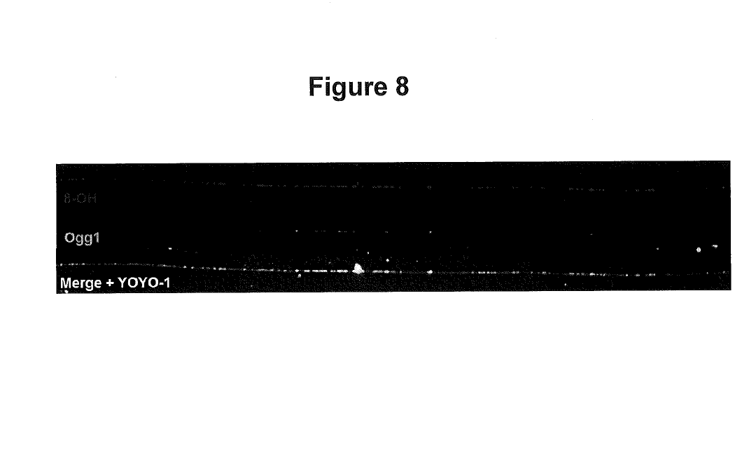

Extended chromatin fibers can be generated from human or animal cells and used to directly detect damaged DNA bases (e.g., 8-Oxo-7,8-dihydro-2′-deoxyguanosine[8-oxo-dG] and cyclobutane dimers [CPDs]). Additionally, chromatin fibers can be utilized to indirectly detect sites of DNA damage by evaluating the fibers for the presence of proteins involved in the detection and repair of DNA damage. A schematic is shown in FIG. 7. For example, extended chromatin fiber analysis can be immunostained for 8-Oxoguanine glycosylase (OGG1; FIG. 8), which is involved in the repair of 8-oxo-dG, ATRIP and phospho-RPA (markers for stalled replication forks often seen after ultra violet [UV] damage), and 53bp1 (a marker for double strand breaks routinely seen with ionizing radiation [IR], but also seen with very high doses of UV).

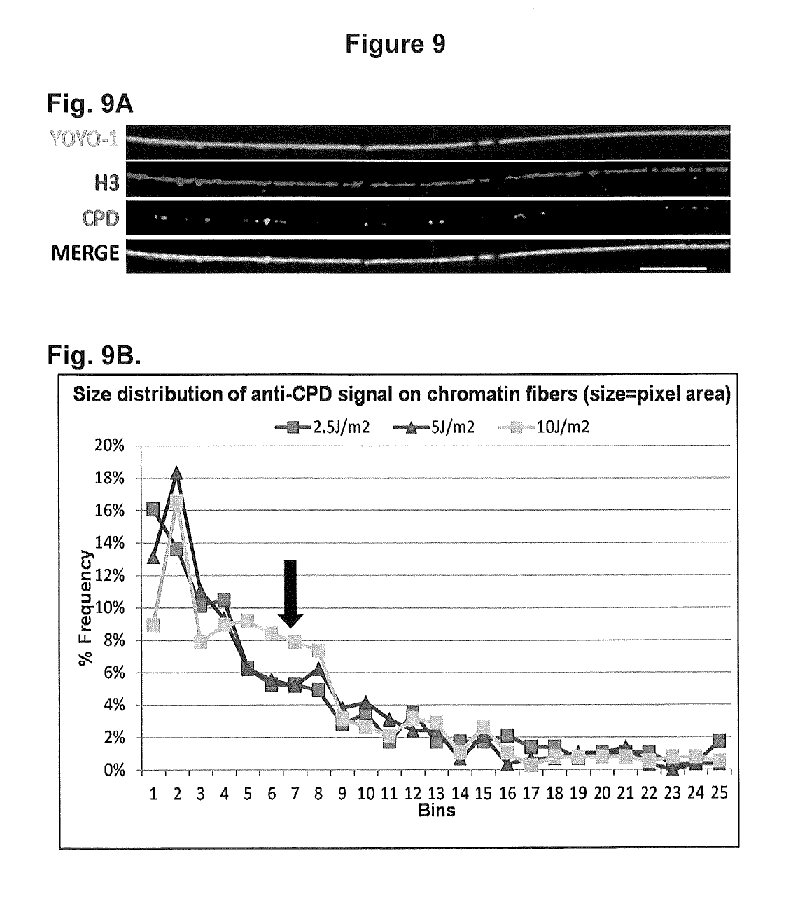

Examples of chromatin fibers stained with two of these proteins after treatment with UVC are shown in FIG. 9. Extended chromatin fibers were generated from normal human fibroblasts (NHF1) as previously described (Cohen et al., Epigenetics and Chromatin 2: 6 (2009)) on Superfrost Plus slides (Fisher Scientific), which contain a positive charge that improves adherence. The lysis buffer was 25 mM Iris, pH 7.5, 0.5 M NaCl, 1% Triton X-100, and 0.2 M urea. First, we used indirect immunofluorescence on chromatin fibers to evaluate the distribution of CPDs on chromatin after exposure to UVC. NHF1 cells were exposed to 2.5, 5, or 10 J/m2 of UVC, collected by trypsinization, processed for extended chromatin fiber analysis and then immunostained for histone H3 and CPDs (an example with 5 J/m2 UVC is shown in FIG. 9, panel A). DNA in the chromatin fibers was stained with YOYO-1, We found an increase in the number of CPDs per megabase (Mb) of DNA but this increase was not linear: a dose of 2.5 J/m² resulted in a density of 3.88 CPD/Mb (13 fibers analyzed, 80 Mb DNA, 314 CPDs), 5 J/m² resulted in a density of 4.0 CPD/Mb (16 fibers, 72 Mb of DNA, 294 CPDs), and 10 J/m2 resulted in a density of 4.6 CPD/Mb (17 fibers, 86 Mb, 395 CPDs). We believe that the lack of a linear dose response was due to the level of resolution of chromatin fibers which is 8.3 fold less than DNA fibers, resulting in an aggregation of antibody signal from closely spaced CPDs (clustering of signal). We therefore analyzed the size of the anti-CPD signals on the fibers to determine whether we were indeed detecting an increase in clustering of CPDs with increased dosage of UVC. The results of this analysis are shown in FIG. 9 (panel B). We found that with a dose of 2.5 J/m² the signal from the anti-CPD most frequently found as single pixels while the most frequently found signal in both 5 J/m² and 10 J/m² samples were doubles. Additionally, the J/m² sample also displayed an increase in the frequency of five, six and seven pixel sized anti-CPD signal (FIG. 9, panel B, arrow). These data confirm a dose-dependent clustering of damage after treatment with UV, which would also be indicative of a non-random distribution of damage from UV.

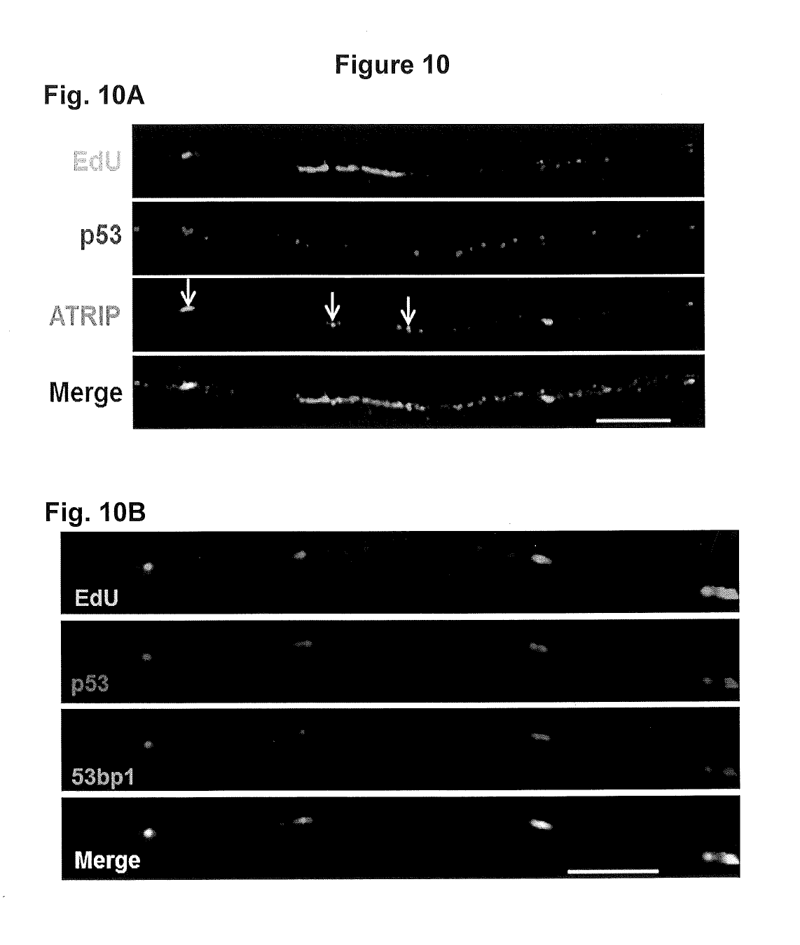

We also used chromatin fibers to study the distribution of ATRIP (and presumably ATR) after UVC treatment, as a marker for stalled replication forks and single stranded DNA after UV damage. For these studies. NHF1 cells were incubated with EdU (a nucleotide analog) for 30 min. Cells were then treated with 10 J/m² UVC and collected either 15 or 45 min post-treatment. Chromatin fibers were prepared and immunostained and EdU was visualized as previously described (Cohen et al., Epigenetics and Chromatin 2: 6 (2009)). FIG. 10 (panel A) shows a representative photomicrograph of chromatin fibers from cells collected 15 min after UVC treatment, with EdU (green signal), p53 (red signal) and ATRIP (blue signal) all present. We found that as expected, there was a high correlation between ATRIP and chromatin fibers with sites of replication (95% chromatin fibers that contained ATRIP also had sites of active DNA replication). Analysis of the distribution of p53 showed a slightly weaker correlation with regions of DNA replication (75% of chromatin fibers that contained p53 also had sites of active replication). We also found that there was a decrease in the levels of chromatin associated ATRIP (down 18%) and an increase in chromatin association of p53 (up 25%) between the 15 min and 45 min time points indicating that ATRIP association with chromatin may be short in duration as compared p53. We have also analyzed the distribution of 53bp1 as a marker of DNA double strand breaks (DSBs). FIG. 10 (panel B) shows a representative photomicrograph of chromatin fibers 2 h after treatment with 20 J/m² UVC with EdU (administered 30 min before UVC treatment), p53 (red signal) and 53bp1 (blue signal).

The distribution of proteins involved in homologous recombination and non-homologous end joining are evaluated to determine the extent of DSBs after UV damage and their relation to sites of active DNA replication.

### Example 5

### Using a Computer-Based

### Method to Detect DNA Damage

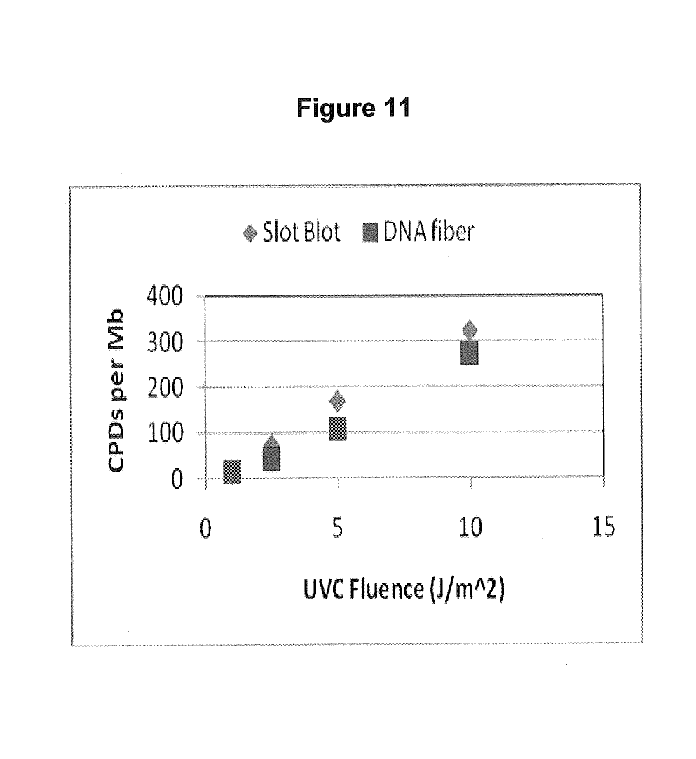

In an effort to improve the sensitivity of detection of DNA damage at the ends of replicating tracks in an unbiased way (i.e., without a person deciding whether a lesion is at the end or not), a program was recently developed that traces individual DNA replications tracks and determines the areas that replicate before damage from the areas that replicated after UV irradiation (Wang et al., Automated DNA Fiber Tracking and Measurement., in Proceedings of the Eighth IEEE international Symposium on Biomedical Imaging: From Nano to Macro (ISBI '11)2011: Chicago, USA), Taking advantage of the program's ability to detect DNA fibers, areas undergoing replication before and after UV radiation, and identify and quantify the amount of DNA damage throughout the aforementioned replication areas, we assessed whether the program and its current algorithm could accurately gauge the amount of DNA damage that occurs after several fluences of UVC. As can be seen in FIG. 11, the program is able to detect DNA damage and the level of damage it detects is similar to what we find using slot blot analysis.

Using this program, we analyzed the number of fibers that contain DNA damage and found there was a dose-dependent increase in the number of fibers that contain DNA damage (Table 1).

TABLE 1

| | | % of fibers | at Ends |
|---|---|---|---|
| UVC fluence | 1 | 25% | 67% |
| | 2.5 | 48% | 69% |
| | 5 | 62% | 67% |
| | 10 | 87% | 81% |

Interestingly, we did not see a dose-dependent increase in the number of fibers that had DNA damage at their ends (Table I). However, we did see that origins that did initiate after DNA damage were more likely to have replication forks encounter lesions (this was also observed for oxidative DNA damage; Example 2). Interestingly, areas replicating after UV damage were 4-6 fold more likely to harbor DNA lesions than areas that replicated before the damage (data not shown). Another interesting finding was that it seems that once a DNA lesion forms in a region that region is more likely to have another lesion form there (Table II).

These multiple lesions are perhaps the main reason why replication forks stop and then collapse. If this is true then there should be more “stalled”/collapsed replication forks at CPDs that are consecutive (clustered). Our initial approach was to detect the number of CPDs at the ends of replication tracks, but the problem with this approach is that it is unable able to distinguish forks that are there simply due the fact that we stopped the reaction at the exact moment in which the replication fork encountered the damage from those forks that are stalled simply as a consequence of them trying to bypass those lesions from those replication forks that are truly stalled/collapsed. Chromatin fiber analysis can be used to distinguish between those various possibilities.

TABLE II
Number of Consecutive CPDs

| | | 1 | 2 | 3 | 4 | 5 | 6 | 7 | 8 | 9 | >=10 Lesions |
|---|---|---|---|---|---|---|---|---|---|---|---|
| UVC | 1 | 51.3% | 27.2% | 11.2% | 3.9% | 2.2% | 1.3% | 0.9% | 1.3% | 0.0% | 0.9% |
| Fluence | 2.5 | 38.6% | 33.0% | 12.1% | 8.7% | 3.4% | 1.9% | 1.1% | 0.0% | 0.4% | 0.8% |
| | 5 | 35.5% | 21.6% | 17.2% | 9.0% | 6.3% | 3.6% | 1.9% | 2.2% | 1.1% | 1.6% |
| | 10 | 20.2% | 15.3% | 12.6% | 9.1% | 6.8% | 6.2% | 4.6% | 3.5% | 3.6% | 18.1% |

### Example 6

### Forming DNA and Chromatin Fibers Using Microfluidics

Microfluidics can be used to form DNA fibers. As one approach, cells are lysed (e.g., as described in Example 1 for DNA fibers or as described in Examples 4 and 5 for chromatin fibers), the DNA purified and both damaged and total DNA fluorescently labeled (e.g., through a DNA intercalator or an antibody that detects DNA that is fluorescently labeled). After labeling the DNA and DNA damage, an aliquot of the sample is placed into an inlet of a microfluidics chamber that straightens the DNA. The straightened DNA is then passed through a fluorescence detector that measures the amount of time a certain fluorescent signal (the one for total DNA) lasts and how many times that signal is interrupted by a different color the fluorescence associated with DNA damage).

As another approach, whole cells are injected into the inlet, and through a number of changes in the shape of the fluidics chamber, the cells are placed into a hypotonic buffer that separates the cell's nucleus from the rest of the cell. The nucleus is lysed using a lysis solution (e.g., as described in Example 1 for DNA fibers or as described in Examples 4 and 5 for chromatin fibers). In the case of DNA fibers, the proteins that are associated with the DNA are stripped off, and the DNA is separated from the proteins using changes in microfluidic flow rates and width of the microfluidic chamber. In the case of either DNA fibers or chromatin fibers, in another inlet(s), a DNA dye and a label for damaged DNA is input into the chamber, and the DNA is incubated with the DNA dye and the label. Afterwards, the DNA undergoes a number of wash steps to remove unbound DNA dye and label. Finally, the DNA is passed through a fluorescence detector to detect total and damaged DNA.

### Example 7

### Detection of DNA Damage Using Micro- and Nanofluidics

Current methods for risk stratification of patients with breast cancer rely on a combination of prognostic factors like tumor size, grade, lymph node status, and presence of hormone receptors (estrogen or progesterone) and the human growth factor receptor 2 (HER-2) and guidelines such as the St. Gallen Consensus Guidelines or Adjuvant! Online. While it is important to classify a patient's type of breast cancer and clinical studies have shown which chemotherapy regimens benefit groups of patients with particular types of tumors, the therapies are not effective for all the individuals in these groups. Treatments based on risk stratification alone are sufficient for a partial response rate of 50 to 60% for most types of breast cancer and a 10-50% complete response with a disease-free survival rate of 50 to 60% after 55 months. It is believed that subtle differences between individual cancers even those of the same classification make each cancer unique and these differences contribute to variations in responses to therapy and disease free survival intervals. Therefore, it would be valuable to have a technique that could be informative about the chemotherapeutic efficacy of a drug or combination of drugs on a tumor in an individual patient. The present invention is design to predict the sensitivity of a patient's cancer to chemotherapeutic agents by quantifying the number of DNA lesions that are generated by those agents in circulating tumor cells (CTCs) from those patients that have an epithelial phenotype, that are undergoing an epithelial-mesenchymal transition (EMT) or that are exhibiting stern cell-like features (CSC). By making such a prediction, it may then be possible to determine which CTC types accumulate excessive amounts of DNA damage from the agents and thus are sensitive to the treatment and which CTC types do not acquire any DNA damage and thus are resistant to the agent. This knowledge is important for being able to better predict which chemotherapy treatment regimen will be the best short and long term option for a cancer patient.

Demonstrating that the Status of Cancer Cells after Chemotherapy Treatment in an In Vitro Setting can Predict Drug Efficacy.

An in vitro test of the capability of the present invention to detect DNA damage induced by chemotherapeutic agents by treating breast cancer cell lines (or a mixed population of breast cancer cells) with chemotherapy agents to which some or all of the cancer cells are susceptible or resistant to the given treatment will be done. Isolated cells that are enriched immunologically for different cell surface markers will be lysed on slides and DNA lesions will be quantified. We will compare untreated cells and cells treated in vitro with drugs used for breast cancer chemotherapy to determine the degree to which they damage DNA and whether the amount of damage is correlated with drug dose.

Demonstrating that the Status of Cancer Cells after Chemotherapy Treatment in Preclinical Breast Cancer Mouse Studies can be Used Predict Drug Efficacy.

A preclinical test will be done of the capability of the present invention to detect DNA damage induced by chemotherapeutic agents in mice bearing xenogransplanted human breast cancer cell lines grown to form solid tumor masses that release circulating tumor cells (CTCs). Several of these human cancer cell lines derived from a common parent line and individual lines manifest different drug resistance characteristics are available. Mice bearing xeno-transplanted human tumor cell lines (or a mixed population of tumor cells) will be treated with drugs to which some or all of the cancer cells are susceptible or resistant to the given treatment. The level of DNA damage in CTCs from these mice will be assessed for a correlation with tumor sensitivity/resistance, the amount of tumor growth/shrinkage or metastasis formation.

Demonstrating that the Status of Cancer Cells after Chemotherapy Treatment in Breast Cancer Patients can Predict Drug Efficacy.

We wish to perform a small, pilot clinical test of the capability of our technology to detect DNA damage induced by chemotherapeutic agents. To validate the relationship between damage induced in the DNA of CTCs from breast cancer patients treated with chemotherapeutic drugs and the clinical response to the treatment of those patients we propose to perform a small proof-of-principle study. If we show that levels of drug-induced DNA damage, cell surface markings, and viability status in a patient's CTCs is highly correlated with the clinical response to the systemically administered drug, we will have achieved a proof-of-principle of our technology to predict the efficacy of an chemotherapeutic agent.

DNA damage in CTCs of treated patients will provide an internal monitor of the efficacy of therapy in each patient. The approach of the present invention will allow processing of large numbers of samples in an efficient manner suitable for a clinical laboratory. In addition, this approach will help to individualize cancer care because it measures effects of therapy in a given patient and this information will allow a clinical oncologist to know which agents are most likely to be effective and which ones will not be. It will also alert a clinical oncologist when a patient's cancer has become resistant to the current treatment thereby allowing the patient to be immediately switched to a new treatment regimen that will be effective.

Methods to Test Potential Therapeutic Agents that Yield Better Predictions of Response.

To understand how an individual cancer patient tumor responds to potential chemotherapy drugs treatments, some cancer researchers are taking a biopsy of a tumor mass, preparing a single cell suspension from the tumor sample, dividing the cells into groups, and exposing the groups of cells to different agents. After the exposure, the number of cells which die due to the treatment is calculated and the amount of cell death caused by the different agents are compared. However, knowing which agent kills the most tumor cells, while useful, does not provide information about the tumor cells that survive and which may give rise to recurrent disease. For instance, do the cells that survive have the capacity to undergo clonal expansion or have they undergone senescence or mitotic catastrophe and thus are unable to expand? Do the surviving cells have the capacity to enter the bloodstream and give rise to metastases? Do they have cell surface markers that distinguish them as stern cells or another aggressive phenotype? Are they resistant to the chemotherapeutic agent or are they sensitive to the agent but require exposure to a higher dose of the agent to be effective?

To circumvent the question about whether the tumor mass cells are capable of entering the bloodstream (and thus potentially causing a metastasis) another approach in use by cancer investigators is to expose circulating tumor cells (CTCs) to therapeutic drugs and determine their effectiveness for killing the CTCs (by looking for a decrease in their number). However, a number of recent published reports suggest that CTC numbers alone may not be sufficient to determine either condition (3-5). Another problem facing researchers who use CTCs is that, for example, in breast cancer patients circulating tumor cells (CTCs) in blood are present in numbers that range from one to hundreds per mL (6). The number of CTCs detected varies with the type and stage of the tumor as well as the efficiency of the technique used for their isolation, i.e., the percentage of the CTCs present in the blood that are recovered [for review, see (7)]. As a result of not being able to obtain a pure population, the “response” of the CTC that researchers obtain may reflect how “normal” cells (white blood cells) are affected by the drug and not the CTCs. Due to the limitations of using only tumor mass cells or CTC, neither method can accurately predict which agent will work best for treating an individual patient's cancer.

In one aspect, the microfluidic based approach of the present invention, which allows us to obtain nearly pure (>90% purity) populations of cancer cells, overcomes many of the inherent short comings of the aforementioned approaches because both tumor mass cell and CTCs are analyzed for their sensitivity towards various chemotherapy agents, thereby giving us an idea of how the primary tumor and potential metastasis respond to the damage. In addition, this approach divides the exposed cancer cells into cells that are undergoing cell death, cells that are alive but cannot most likely undergo clonal expansion, and ones that are alive and can expand. Further, our method subdivides these on cells based on whether they express Epithelial cell adhesion molecule (EpCAM), are undergoing a epithelial-mesenchymal transition (EMT, expressing N-cadherin), or are exhibiting stem cell-like features (CSCs, expressing CD44+) (8-10). EMT induction in cancer cells results in the acquisition of invasive and metastatic properties while CSC promotes self-renewal (11).

The present invention can then analyze the amount of DNA damage that was caused by the chemotherapeutic in the various subpopulations of cancer cells. If a cell is sensitive to a chemotherapeutic agent, it will accumulate lots of DNA damage (e.g., oxidative DNA damage, double-strand breaks, cisplatin damage, and the like) and conversely if it has little or no damage (above its normal background level) then it is resistant to the drug. By looking at the damage in the cells that are alive and potentially could undergo clonal expansion, it will be possible to determine whether these cells are resistant to the drug treatment (because they do not have elevated levels of damage after treatment) or are sensitive but did not receive enough of the agent to kill them or cause them to undergo senescence or mitotic catastrophe.

The ability to quantify DNA damage in the CTCs from a few milliliters of peripheral blood would make it possible to monitor frequently the effects of many chemotherapeutic drugs administered to a patient. Such measurements may permit the discovery of resistance to the drug soon after resistance develops and before recurrent tumor is detectable by radiological imaging techniques. It may also be possible to analyze resistant CTCs for sensitivity to a another chemotherapeutic drug by treating the resistant CTCs with new drugs in vitro to identify drugs to which the tumor is sensitive. We see the development of this process to determine the sensitivity of CTCs to chemotherapeutic drugs to be part of the effort to change the paradigm of cancer therapy from one using past success with a particular drug in a group of patients with similarly classified tumors, to one that employs “personalized medicine” in therapeutic decision-making in the clinical care of individual cancer patients. This method permits the choice of drugs based on knowledge of the efficacy of the drug in a particular patient's CTCs.

The methods of detection of DNA damage of the present invention will enable clinical oncologists greater capacity to predict how a patient's tumor mass and CTCs will respond to a particular treatment regimen. This new information will increase the likelihood of a therapeutic response at least four to eight times (12), detect the onset of drug resistance, and decrease potential side effects by determining minimal effective doses. It will also have the capability to test the effect of multiple agents together to determine whether a multidrug treatment regimen is better than a single agent. In just a few hours' time, this approach will allow the determination of which potential therapeutic agents will have a greater than 80% chance of therapeutic efficacy in a particular individual patient's tumor and distinguish them from agents that will not work and thus, should yield better predictions of therapeutic responses than that which is currently available.

One method for detecting DNA damage described herein uses a multi-step procedure to identify and quantify DNA damage. This approach can be used to analyze 100 samples per day per clinical laboratory technician and will be appropriate for the pilot-study. Another method described herein uses a single system providing full process automation of the multi-step assay to search for CTCs, isolate their genomic DNA using a solid-phase extraction technique and then, search their genomic DNA for the AP sites using a nanosensor (See, e.g., FIG. 17). This latter method should make DNA damage measurements for CTCs suitable for performance in a clinical laboratory setting with rapid (1 to 2 hours) turn-around time, high throughput (hundreds of samples per day), and with limited clinical laboratory technician effort. This system can be designed to use a unique architecture, in which task-specific modules made from a thermoplastic or other material are poised on a fluidic motherboard. A nanosensor module (See, e.g., FIG. 17) can employ an electrical readout strategy to identify AP sites without requiring immobilization of the target DNA or optical hardware; this electrical readout should be able to generate exquisite spatial resolution, All of these modules can be produced at low-cost and a high production mode to facilitate their usage in clinical settings. The ability to obtain these results quickly and efficiently and at low-cost should facilitate the adoption of this test in personalized cancer care scenarios.

Detection of DNA Damage Caused by a Chemotherapy Agent

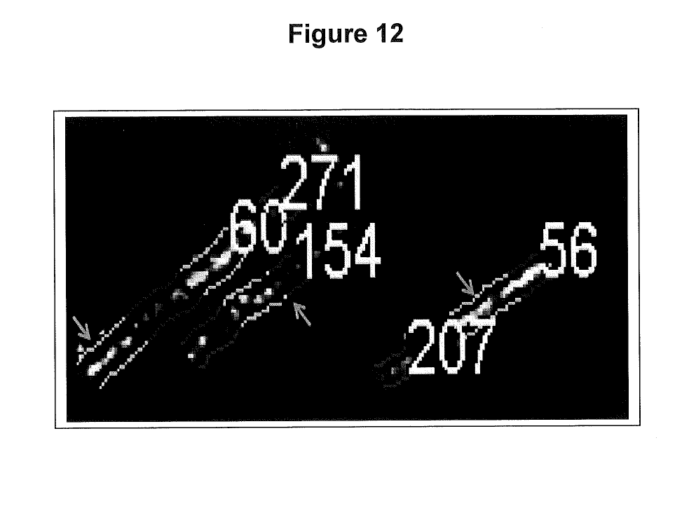

The methods of the present invention detect DNA damage (AR sites) in isolated genomic DNA by tagging them with fluorescent-ARP (FARP) and visualizes them by confocal laser scanning microscopy (CLSM) (13), Over 10⁹-nucleotides (nt) of DNA were analyzed and 5.4 AP sites per 10⁶ nt were detected versus the 5.8 detected by the slot blot method (FIGS. 1, 2 and 5). DNA damage in DT40 cells was similar to that found in HeLa cells and calf thymus DNA (14). When 7×10⁸ nt of DNA from cells exposed to 20 μM H₂O₂ in vitro was analyzed, the number of AP sites was found to have increased to about 8 AP sites per 10⁶ nt (which resemble results of slot blot analysis in FIGS. 1, 2 and 5). In both untreated and treated cells, AP sites were observed to occur in clusters. The number of AP sites per 10⁶ nt found by the fiber spreading approach is within 10% of the value determined by slot blot analysis. In addition, method of the present invention accurately determines the number of AP sites under both normal tissue culture and oxidative stress conditions. Further, the staining of DNA fibers and acquisition of digital images of the fluorescent DNA fibers and DNA damage can be automated. A program has been developed that detects fibers in images (FIG. 12), measures the fibers and detects and quantifies DNA damage.

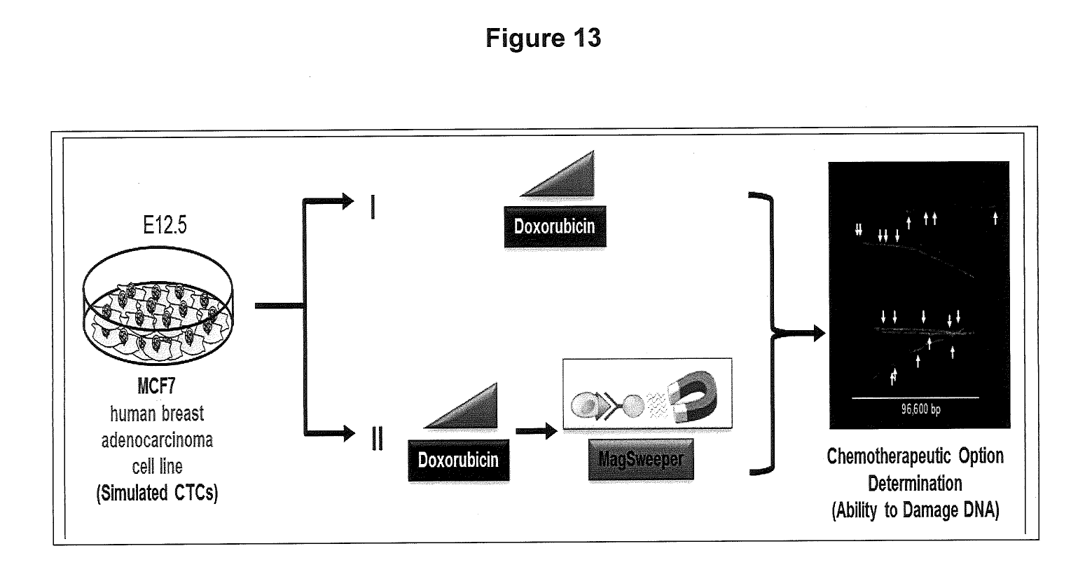

In an initial study, MCF7 human breast cancer cells, a line of human breast adenocarcinoma, were used to simulated CTCs. The “simulated CTCs” were exposed to 0, 0.33, and 0.66 μM doxorubicin (Dox). Doxorubicin is a principal chemotherapeutic agent for breast cancer and the doses of the drug were chosen to approximate dose levels achieved in patients. In one experiment, untreated cells and cells treated with Dox were harvested 16 hours after starting sham or drug treatment and slides of extended DNA were prepared from the untreated and treated cells. The amount of DNA damage for each treatment condition was then determined. This experiment was designed to test whether drug exposure of the cells alone increased DNA damage in the form of abasic (AP) sites (FIG. 13, upper path). Next, untreated cells and cells treated with Dox were collected 16 hours after starting sham or drug treatment. The cells were subjected to a CTC isolation technique referred to as the ‘Mag Sweeper’ process (15) and the recovered cells' DNA harvested and placed on slides to extend the DNA and then the DNA analyzed for damage as described herein (FIG. 3, lower path). This experiment showed that the Mag Sweeper technology, itself, did not induce any DNA damage and that the DNA damage caused by the Dox treatment was detectable.

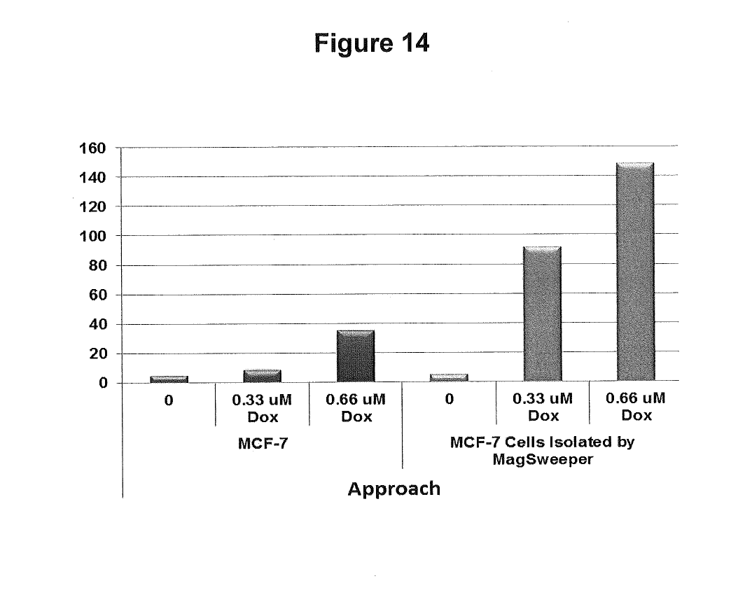

The results of this study showed that MCF-7 cells responded to Dox with notably increased DNA damage [6] (FIG. 14). The level of AP sites in the MCF-7 cells was higher after treatment with Dox than in untreated cells (FIG. 14). When Dox-treated cells and untreated control cells were processed using the Mag Sweeper, recovered the MCF-7 cells (FIG. 14) were assessed for DNA damage. This study used surrogate CTCs that simulate the recovery of CTCs from Dox-treated patients and from untreated patients, or from the same patient before and after Dox treatment. Again, AP sites increased after Dox treatment, but in this case more DNA damage was observed. In both studies DNA damage in controls was very low. There also was more damage detected with the higher dose in both cases. The analysis of DNA damage took 1 person about 4 hours to stain, process, and analyze all the slides, 36 in total, needed for this study.

Demonstrating that DNA Damage Quantified in Small Numbers of CTCs can Predic Drug Efficacy in In Vitro Studies.

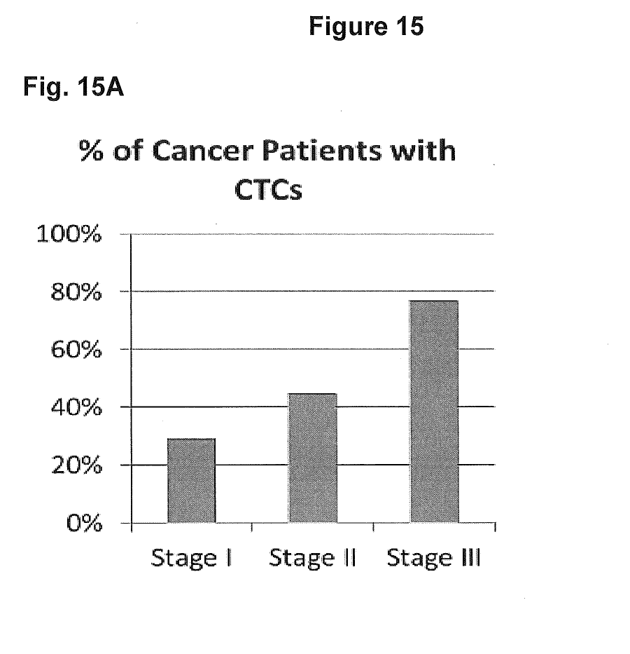

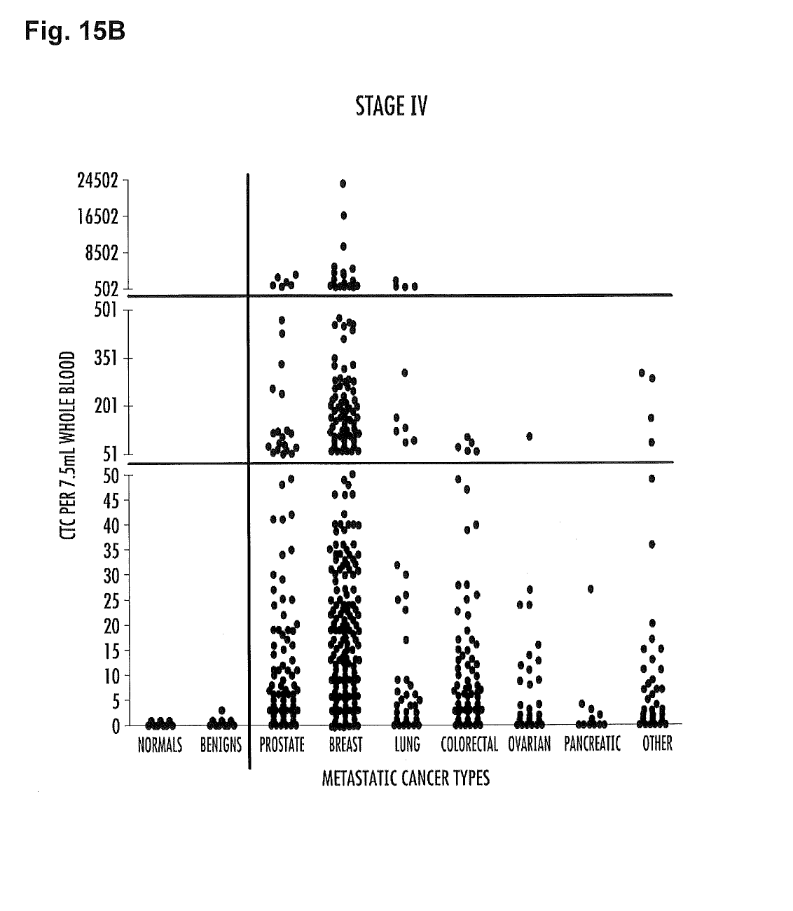

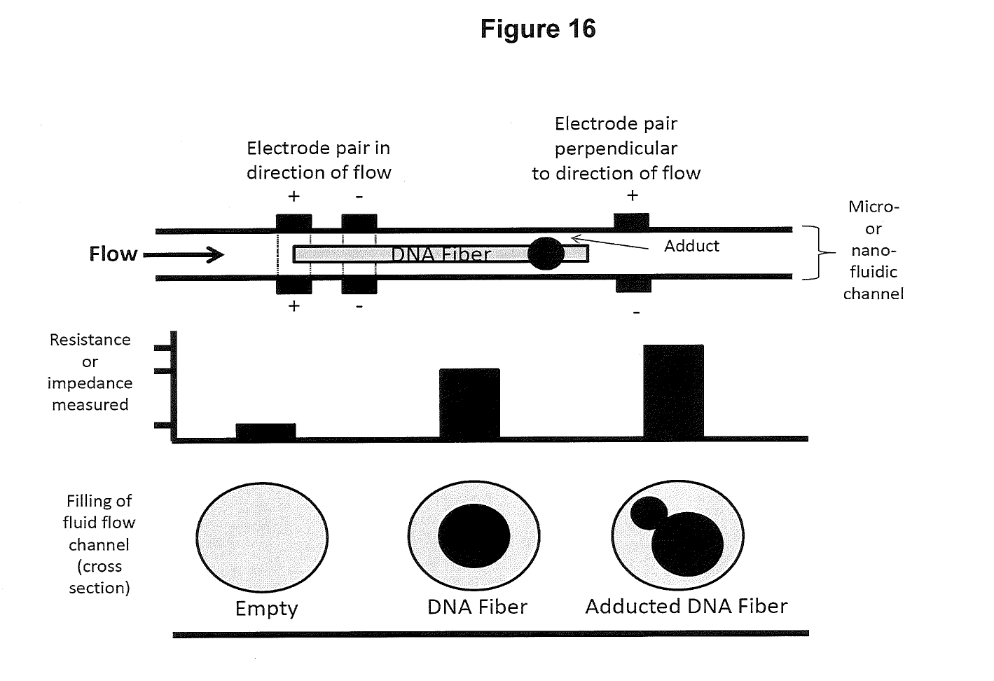

Invasive growth by malignant solid tumors like breast cancers facilitates access of their cells to underlying blood vessels and is often associated with the appearance of cancer cells that circulate in the blood stream and formation of metastases. CTCs are thought to reflect the genetic and phenotypic diversity of the tumor and evolve in a manner reflecting the progression of the primary tumor and metastases (for review, see (7)). Thus CTCs may serve as surrogates for a patient's solid tumors and may have the potential to predict the sensitivity of a patient's cancer to various chemotherapeutic agents. CTCs can be found in patients in each stage of breast cancer; patients with advanced stage cancers have the greatest likelihood of producing them (FIG. 15A; (2)). In addition, CTCs can be found in patients with all major cancers but not in healthy subjects or patients with nonmalignant diseases (FIG. 15B; (1)). Therefore, CTCs may be available in a large proportion of women with breast cancer and in sufficient numbers to assess chemotherapy-induced DNA damage accurately using our DNA fiber approach where damage sites are visualize in extended DNA fibers.

DNA Damage for Predicting the Efficacy of a Potential Cancer Treatment:

Of the 24 primary therapeutic agents currently in use, about 50% induce abasic (or AP) sites and an additional 40% produce double strand breaks (DSBs) (16-42). Because most chemotherapy drugs cause DNA damage, either directly or indirectly, then DNA damage in CTCs caused by the drug in vivo may provide a measure of the drug's effectiveness. The benefit of using CTCs over the primary or metastatic site(s) for DNA damage assessment is that CTCs allow for frequent monitoring that can be prohibitive using tissue biopsies, even fine-needle aspiration biopsies. DNA damage assessment methods that indirectly determine damage, such as gamma H2AX foci formation or the comet assay, have not been effective at predicting the efficacy of a chemotherapy drug in cancer patients (3). The most likely reasons are that these methods are not usable within hours after treatment (43-46), can severely overestimate the degree of damage and/or may not be sensitive enough (47).

If DNA damage is to be used as a measure of drug efficacy, then it is necessary to be able to accurately quantify the amount of DNA damage in a limited number of CTCs. We have developed a method to assess DNA damage by extending DNA fibers on a microscope slide, fluorescently labeling DNA and sites of DNA damage on the extended DNA fibers, and enumerating sites of DNA damage per unit-length of DNA by image analysis. This technique enables us to identify DNA damage from a small number of cells and it yielded statistically significant findings from 5 rather than millions of cells. This method was shown to yield results as accurate as those produced by the slot blot assay that is the “gold standard” for measuring abasic sites, a type of oxidative DNA damage in DNA, and our technique does this with lower variance (i.e., greater precision) than do the slot blots (13). Our method can measure any type of DNA damage for which there is an antibody or probe for that damage. Recently, we confirmed that our method is capable of detecting DNA damage caused by doxorubicin (See. FIG. 14).

### Materials and Methods:

Cell Lines:

MCF-7 (doxorubicin and paclitaxel sensitive, estrogen and progesterone receptor positive), MCF-7-Dox (estrogen and progesterone receptor positive, doxorubicin resistant), MCF-7-Tax (estrogen and progesterone receptor positive, paclitaxel resistant), HCC1937 (estrogen and progesterone receptor, negative. BRCA1-defective, cisplatin sensitive), MDA-MB-231 (hormone-insensitive, triple negative (estrogen and progesterone receptor negative, Her2/neu negative]), Hs 578T (high CD146 and CD44 expression and low CD24), and HCC38 (estrogen and progesterone receptor, negative; 100% of the cells are cancer stem cells (CSCs)(48)) cell lines will be used as CTC surrogates in this study. MCF-7 was chosen because 70 to 80% of primary breast cancers are estrogen receptor (ER) and/or progesterone receptor (PR) positive (49). In addition, up to 1% of MCF7 cells are Cancer Stem Cells (48). MCF-7-Dox and MCF-7-Tax were chosen because most women whose cancer are sensitive (about 40% of estrogen and/or progesterone receptor positive breast cancers are sensitive) to Dox or Paclitaxel become resistant to treatment at some point (50-52) and b) this resistance is most likely due to the cancer cells increasing their antioxidant capacity to counteract the oxidative stress caused by Dox or Paclitaxel (53). HCC1937 was selected because germline mutations of the tumor suppressor gene BRCA1 are involved in the predisposition and development of breast cancer and account for 20-45% of all hereditary cases (54). MDA-MB-231 was chosen because about 15% of breast cancers are triple negative (55). MDA-MB-231, along with Hs 578T, has high fibroblast activation protein alpha expression (FAP alpha) and CD44 and low CD24 expression and are considered to EMTs (9), HCC38 was chosen because it contains a near pure population of cancer stem cells (48) and because it has been possible to produce viable breast cancer xenografts by implanting these cell lines into mouse mammary fat pads (56-59).

Chemotherapy Agents:

The following agents will be used; doxorubicin, paclitaxel, cisplatin, and tamoxifen. Doxorubicin is one of the most common chemotherapy treatment options for breast cancer (60). Paclitaxel is used for hormone-insensitive cancer (61-64), and to eradicate occult micrometastatic disease in the adjuvant setting. Cisplatin is used as; 1) a second line of treatment once a breast cancer patient has acquired resistance to the initial treatment option, 2) as a treatment option in BRCA1-associated cancers (65, 66), and 3) as a neoadjuvant (65). Tamoxifen is often the treatment option of choice for estrogen receptor positive breast cancer (40-42). Three of these agents (doxorubicin, paclitaxel, and tamoxifen) produce AP sites and one produces a DNA crosslink (cisplatin) (26, 43, 61, 63-65, 67-69), all of which can be detected with the DNA damage detection methods of this invention. In addition, the formation of DNA damage by these agents inside a cancer cell is one of the main modes of action by which these agents are effective and resistance to these agents is correlated with a cancer's ability to avoid DNA damage formation by these agents (17, 19, 21, 24, 26, 28, 31, 33, 34, 38, 39), Together these treatment options simulate various chemotherapy choices available to an oncologist.

The methods of the present invention provide automated high throughput system for detecting DNA lesions using a nanosensor, which is comprised of a single nanochannel containing one or two pairs of nano-electrodes. The electrodes will measure the presence of DNA lesions with or without labeling of the lesions (e.g., labeling with an Aldehyde Reactive Probe (ARP) bearing a biotin molecule and subsequently complexed with streptavidin to facilitate detection using a single-molecule electrical readout).

A nanosensor module for directly reading lesions sites in genomic DNA can incorporate transverse electrodes, Determining the presence and location of sequence variants using optical techniques following DNA elongation, can be limited due to diffractions effects (about λ/2 (about 250 nm, which can be improved by using super resolution techniques, such as STEAD). Therefore, in some embodiments of this invention, the methods transduce the presence of DNA lesions sites using nano-scale electrodes, which can provide resolution that can be determined by scaling effects and is related to the size of the electrodes.

As an initial evaluation of the methods of this invention, AP sites using standard dsDNAs with known lengths and AP sites (positive control) or no AP sites (negative control) can be measured as a model system (see, for example, schematic in FIG. 17). In some aspects, the AP sites can be labeled with an aldehyde reactive probe (ARP) that bears a biotin molecule. Following labeling with ARP, the dsDNA can be incubated with unlabeled streptavidin, which forms stable complexes with biotin. The electrical response produced by the sensor(s) can provide information as to the length of single DNA molecules and the relative positions of the AP sites within the target DNA (89). In addition, different gaps between the nano-electrodes can be produced to improve the signal-to-noise ratio in transducing AP sites that are signaled by the presence of the streptavidin molecule bound to the biotinylated ARPs. The unique architecture of the design of this invention should mitigate any problems that may arise from folding conformations of dsDNAs that can complicate the analysis (94). This consists of an array of nanopillars that slightly unravel the DNA before entering the nanochannel. In addition, tapered inputs for the single nanochannel enhance the feed rate of DNAs into the nanochannel (95). The use of the dual electrodes provides a further advantages by providing better identification efficiency of the presence of AP sites; cross-correlation analysis of signals generated at the two pairs of electrodes and by improving the identification even under conditions when the signal-to-noise ratio is low.

In an exemplary embodiment, current transients can be composed of two components: (1) A constant current perturbation indicative of the DNA molecule passing through the electrodes arising from ion displacement changing the conductance of the interstitial space between the electrodes. This constant transient will be correlated to the length of the DNA molecule (93, 94); and/or (2) A spike(s) in the transient arising from the presence of the streptavidin molecule due to additional ion displacement from volume exclusion effects imposed by the presence of streptavidin (˜620 nm³; 6×6×18 nm) and its characteristic specific ionic conductance. To mitigate artifacts related to potential non-specific adsorption to the channel wall, especially in the case of streptavidin, the nanochannels can be coated with a lipid bilayer (96).

An advantage of using transverse nano-electrode geometries as opposed to optical readout strategies for mapping sequence variations in target DNAs is that the diffraction barrier is non-operable in this case. The positional resolution, which is related to the location of the sequence variant in bp, is determined by the size of the nano-electrodes and the degree of elongation of the target DNA molecule. Additionally, electrical readout does not require labeling of the DNA strand with an intercalating dye, distinguishing this emission from the reporter of the sequence variant and finally, bleaching of the reporters does not occur in the case of electrical readout.

Demonstrating that DNA Damage Quantified in CTCs Predicts Drug Efficacy in Preclinical Breast Cancer Mouse Studies.

The predicted response to the drug based on the amount of DNA damage in CTCs will be compared to the actual response in a mouse tumor xenograft model. Initially, we will show that the chemotherapeutic agent that would be chosen based on having caused the most DNA damage in the CTCs will be the agent that causes the most damage to the DNA in the tumor cells. This will establish the connection between our measures of the CTCs and the tumor cells. In addition, the amount of DNA damage quantified in CTCs harvested and treated in vitro at the outset will be analyzed for its ability to predict the probability of an objective tumor response.

### Material and Methods.

Mice:

Inbred strains of immunocompromised mice that accept human tumor xenografts, and have a uniform genotype will be used for control and experimental conditions. Female NOD SCID mice (NOD.CB17-Prkdc^{scid}/J from the Jackson Laboratory) will be used as they lack T cells, B cells, and are leaky for NK cells.

### Estrogen Pellet Implantation:

This procedure will be performed one week prior to mice receiving breast cancer cells that are estrogen receptor positive.

Development and Treatment of Pre-Clinical Mouse Models:

Each cancer cell line will be injected into the mammary fat pads. Nine mice will be used for each part. The mice will then be followed for tumor growth. The first set will be sacrificed when the tumor is approximately 45 mm. Both CTCs and tumor cells will be harvested. These cells will be isolated and treated with several different chemotherapeutic agents and the amount of DNA damage due to each agent will be quantified, We will determine if the agent that would have been chosen based on having produced the most DNA damage in the CTCs is also the agent that produces the most DNA damage in the corresponding tumor cells.

The other set of mice will be injected with one of the chemotherapy agents with clinically relevant doses and treatment cycles (e.g., 5 mg/kg body weight of Dox once a week for four weeks) beginning when the tumors reach approximately 45 mm. During this time, CTC number per mL of blood and tumor size will be measured, and the amount of DNA damage in the CTCs will be quantified. At the end of the treatment cycle, the mice will be sacrificed and CTCs and tumor tissue will be analyzed for AP levels and tumor size. The objective tumor response will be compared to the damage in CTCs that had been treated with chemotherapeutic agents in vitro at the outset.

### Statistical Methods:

Tumor Response.

For individual mice, progressive disease (PD) will be defined as <50% regression from initial volume during the study period and >25% increase in initial volume at the end of the study period. Stable disease (SD) will be defined as <50% regression from initial volume during the study period and ≦25% increase in initial volume at the end of the study. Partial response (PR) will be defined as a tumor volume regression≧0.50% for at least one time point but with a measurable tumor mass (≧0.10 cm³). Complete response (CR) will be defined as a disappearance of measurable tumor mass (tumor volume<0.10 cm³) for at least one time point. A CR will be considered maintained (MCR) if the tumor volume was <0.10 cm³ at the end of the study period. Treatment groups with PR, CR, or MCR will be considered to have had an objective response. Agents inducing objective responses are considered highly active against the tested agent, while agents inducing SD or PD are considered to have low level of activity against the control group.

DNA Damage Formation

To estimate the effect of chemotherapeutic agents on DNA damage formation in DNA fibers globally, a Poisson regression will be used to model the distribution of AP sites. A Wald test will be used to determine the statistical significance of the chemotherapeutic agent effect.

Chemotherapy Efficacy Prediction

A reduction in tumor size, be it PR or CR, will be considered a positive predictive value (PPV). An increase in DNA Damage Levels, at least two times the average of the control group, will be considered a PPV. PPVs from two different predictive models will be tested for agreement using Fisher's exact test. A p value of <0.05 will be considered to significantly predict clinical outcome (tumor reduction). CTCs are expected to be isolated that are at least 80% pure, which display a similar sensitivity to a chemotherapeutic agent, as indicated by an increase in AP site number, as tumor cells located in the primary tumor. It is also expected that the fold increase in AP site number will directly correlate with how effective a drug will be in reducing CTC numbers and tumor size.

Demonstrating Quantification of Drug-Induced DNA Damage in Patients' CTCs Predicts Therapeutic Efficacy of the Drugs in Patients.

In this study, a patient group will be chosen in which CTCs have a high likelihood of being present because of the stage of the tumor and the tumor mass present, and in which chemotherapy of the patients has a high probability of having an objective clinical response to treatment, such as a measurable reduction of the tumor size in a short time interval (weeks to months).

Material and Methods:

Patients will be evaluated who present with advanced stage disease with reasonably high tumor burden (e.g. non oligometastasis) and who are about to embark on a new chemotherapy regimen. Patients will be recruited from the breast cancer services at Stanford and the University of North Carolina Hospitals, and baseline CTC will be quantitated. Based on prior studies, measurable CTC is expect to be present in over 50% of the breast cancer patients (98). Blood samples will be obtained from these patients before and 1-2 weeks after the start of their chemotherapy and DNA damage in their CTCs, if CTCs are present, will be quantified to determine whether or not there is a statistically significant increase in their DNA damage following treatment. These patients will be assessed as part of their standard-of-care follow-up to ascertain whether the therapy has had a clinically beneficial response, The results of follow-up clinical procedures performed on each patient to determine clinical responsiveness to chemotherapy will be evaluated in comparison to the DNA damage quantified in their CTCs before and after the start of chemotherapy. If comparable increases are seen in CTCs from patients before and after drug treatment, a biostatistical consultant made preliminary power-function estimate suggesting that significance can be achieved with a cohort size from as few as ten patients who have recoverable CTCs. Evaluation of drug-induced damage in the DNA of CTCs will be done by quantifying the number of DNA damage lesions present per 10⁶ nucleotides of DNA. A statistical analysis of the results of this study will determine whether a statistically significant increase in the level of DNA damage in CTCs correlates with clinical response to therapy and a lack of statistically significant increase in the level of DNA damage in CTCs correlates with the absence of a clinical response to therapy. Robust increases have been observed in DNA damage quantified from cultured breast cancer cells treated with chemotherapeutic drugs versus their untreated controls. On this basis, it is expected that meaningful increases of DNA damage in CTCs will be detected from the same patients after versus before therapy.

The foregoing is illustrative of the present invention, and is not to be construed as limiting thereof. The invention is defined by the following claims, with equivalents of the claims to be included therein.

### REFERENCES FOR EXAMPLES 1-6

1. Cooke, M. S., Evans, M. D., Dizdaroglu, M., and Lunec, J. (2003) Oxidative DNA damage: mechanisms, mutation, and disease. Faseb J 17, 1195-1214
2. Chastain, P. D., 2nd, Nakamura, J., Swenberg, J., and Kaufman, D. (2006) Nonrandom AP site distribution in highly proliferative cells, Faseb J 20, 2612-2614
3. Liu, Y. Kao, H. I., and Bambara, R. A. (2004) Flap endonuclease 1: a central component of DNA metabolism. Annu Rev Biochem 73, 589-615
4. Nakamura, J., and Swenberg, J. A. (1999) Endogenous apurinic/apyrimidinic sites in genomic DNA of mammalian tissues. Cancer Res 59, 2522-2526
5. Gill, R., Tsung, A., and Billiar, T. (epub Jan. 20, 2010) Linking oxidative stress to inflammation: Toll-like receptors. Free Radic Biol Med
6. Misra, M. K., Sarwat, M., Bhakuni, P., Tuteja, R., and Tuteja, N. (2009) Oxidative stress and ischemic myocardial syndromes, Med Sci Monit 15, RA209-219
7. Bozner, P., Grishko, V., LeDoux, S. P., Wilson, G. L. Chyan, Y. C., and Pappolla, M. A. (1997) The amyloid beta protein induces oxidative, damage of mitochondrial DNA. J Neuropathol Exp Neurol 56, 1356-1362
8. Multhaup, G., Ruppert, T., Schlicksupp, A., Hesse, L. Beher, D., Masters, C. L., and Beyreuther, K. (1997) Reactive oxygen species and Alzheimer's disease, Biochem Pharmacol 54, 533-539
9. Scapagnini, G., Caruso, C., Vasto, S., Pascale, A., Romeo, L. D'Agata, V., Intrieri, M., Sapere, N., and Li Voilti, G. (2010) Genetic risk factors and candidate biomarkers for Alzheimer's disease. Front Biosci (Schol Ed) 2, 616-622
10. Barber, S. C., and Shaw, P. J. (2010) Oxidative stress in ALS: key role in motor neuron injury and therapeutic target. Free Radic Biol ivied 48, 629-641
11. Mukherjee, S. K., and Adams, J. D., Jr. (1997) The effects of aging and neurodegeneration on apoptosis-associated DNA fragmentation and the benefits of nicotinamide. Mol Chem Neuropathol 32, 59-74
12. Radunovic, A., Porto, W. G., Zeman, S., and Leigh, P. N. (1997) Increased mitochondrial superoxide dismutase activity in Parkinson's disease but not amyotrophic lateral sclerosis motor cortex. Neurosci Lett 239, 105-108
13. Miller, R. L., James-Kracke, M., Sun, G. Y., and Sun, A. Y. (2009) Oxidative and inflammatory pathways in Parkinson's disease. Neurochem Res 34, 55-65
14. Halliwell, B. (1992) Reactive oxygen species and the central nervous system. J Neurochem 59, 1609-1623
15. Alexander, R. W. (1998) Atherosclerosis as disease of redox-sensitive genes. Trans Am Clin Climatol Assoc 109, 129-145; discussion 145-126
16. Fiorillo, C., Oliviero, C., Rizzuti, G., Nediani, C., Pacini, A., and Nassi, P. (1998) Oxidative stress and antioxidant defenses in renal patients receiving regular haemodialysis. Clin Chem Lab Med 36, 149-153
17. Singh, U., and Jialal, I. (2006) Oxidative stress and atherosclerosis. Pathophysiology 13, 129-142
18. De Rosa, S., Cirillo, P., Paglia, A., Sasso, L. Di Palma, V., and Chiariello, M. (2010) Reactive oxygen species and antioxidants in the pathophysiology of cardiovascular disease: does the actual knowledge justify a clinical approach? Curr Vasc Pharmacol 8, 259-275
19. Tissie, G., Guillermet, V., Latour, E., Coquelet, C. and Bonne, C. (1988) Oxidative stress and lens opacity: an overall approach to screening anticataractous drugs. Ophthalmic Res 20, 27-30
20. Varma, S. D., Devamanoharan, P. S., and Morris, S. M. (1995) Prevention of cataracts by nutritional and metabolic antioxidants. Crit. Rev Food Sci Nutr 35, 111-129
21. Varma, S. D., and Devamanoharan, P. S. (1995) Peroxide damage to rat lens in vitro: protective effect of dehydroascorbate. J Ocul Pharmacol Ther 11, 543-551
22. Berthoud, V. M., and Beyer, E. C. (2009) Oxidative stress, lens gap junctions, and cataracts. Antioxid Redox Signal 11, 339-353
23. Nicolas, M, G., Fujiki, K., Murayama, K., Suzuki, M. T., Shindo, N., Hotta, Y., Iwata, F., Fujimura, T., Yoshikawa, Y., Cho, F., and Kenai, A. (1996) Studies on the mechanism of early onset macular degeneration in cynomolgus monkeys. H. Suppression of metallothionein synthesis in the retina in oxidative stress. Exp Eye Res 62, 399-408
24. Plafker, S. M, (2010) Oxidative stress and the ubiquitin proteolytic system in age-related macular degeneration. Adv Exp Med Biol 664, 447-456
25. Stadtman, E. R., and Berlett, B. S. (1998) Reactive oxygen-mediated protein oxidation in aging and disease. Drug Metab Rev 30, 225-243
26. Beckman, K. B., and Ames, B. N. (1998) The free radical theory of aging matures. Physiol Rev 78, 547-581
27. Kujoth, G. C., Hiona, A., Pugh, T. D., Someya, S., Panzer, K. Wohlgemuth, S. E., Hofer, T., Seo, A. Y., Sullivan, R., Jobling, W. A., Morrow, J. D., Van Remmen, H., Sedivy, J. M., Yarnasoba, T., Tanokura, M., Weindruch, R., Leeuwenburgh, C., and Prolla, T. A. (2005) Mitochondrial DNA mutations, oxidative stress, and apoptosis in mammalian aging. Science 309, 481-484
28. Finkel, T., and Holbrook, N. J. (2000) Oxidants, oxidative stress and the biology of ageing. Nature 408, 239-247
29. Vuillaume, M. (1987) Reduced oxygen species, mutation, induction and cancer initiation. Mutat Res 186, 43-72
30. DeWeese, T. L. Shipman, J. M., Lanier, N. A., Buckley, N. M., Kidd, L. R., Grooprnan, J. D., Cutler, R. G., te Riele. H., and Nelson, W. G. (1998) Mouse embryonic stem cells carrying one or two defective Msh2 alleles respond abnormally to oxidative stress inflicted by low-level radiation. Proc Natl Aced Sci USA 95, 11915-11920
31. Halliwell, B. (2007) Oxidative stress and cancer: have we moved forward? Biochem J 401, 1-11
32. Ralph, S. J., Rodriguez-Enriquez, S., Neuzil, J., Saavedra, E., and Moreno-Sanchez, R. (2010) The causes of cancer revisited: “mitochondrial malignancy” and ROS-induced oncogenic transformation—why mitochondria are targets for cancer therapy. Mol Aspects Med 31, 145-170
33. Nowsheen, S., Wukovich, R. L. Aziz, K., Kalogerinis, P. T., Richardson, C. C., Panayiotidis, M. I., Bonner, W. M., Sedelnikova, O. A., and Georgakilas, A. G. (2009) Accumulation of oxidatively induced clustered DNA lesions in human tumor tissues. Mutat Res 674, 131-136
34. Bennett, P., Ishchenko, A. A., Laval, J., Paap, B., and Sutherland, B. M. (2008) Endogenous DNA damage clusters in human hematopoietic stern and progenitor cells. Free Radic Biol Med 45, 1352-1359
35, Bennett, P. V., Cuomo, N. L. Paul, S., Tafrov, S. T., and Sutherland, B. M. (2005) Endogenous DNA damage clusters in human skin, 3-D model, and cultured skin cells. Free Radio Biol Med 39, 832-839
36. Sutherland, B. M., Bennett, P. V., Cintron, N. S., Guida, P., and Laval, J. (2003) Low levels of endogenous oxidative damage cluster levels in unirradiated viral and human DNAs. Free Radic Biol Med 35, 495-503
37. Pastukh, V., Ruchko, M., Gorodnya, O., Wilson, G. L., and Gillespie, M. N. (2007) Sequence-specific oxidative base modifications in hypoxia-inducible genes. Free Radic Biol Med 43, 1616-1626
38. Buerstedde, J. M., and Takeda, S. (1991) Increased ratio of targeted to random integration after transfection of chicken B cell lines. Cell 67, 179-188
39, Matsuzaki, Y., Adachi, N., and Koyama, H. (2002) Vertebrate cells lacking FEN-1 endonuclease are viable but hypersensitive to methylating agents and H₂O₂. Nucleic Acids Res 30, 3273-3277
40. Herrick, J., and Bensimon, A. (1999) Single molecule analysis of DNA replication. Biochimie 81, 859-871
41. Lebofsky, R., and Bensimon, A. (2003) Single DNA molecule analysis: applications of molecular combing. Brief Funct Genomic Proteomic 1, 385-396
42. Jackson, D. A., and Pombo, A. (1998) Replicon clusters are stable units of chromosome structure: evidence that nuclear organization contributes to the efficient activation and propagation of S phase in human cells. J Cell Biol 140, 1285-1295
43. Merrick, C. J., Jackson, D., and Diffley, J. F. (2004) Visualization of altered replication dynamics after DNA damage in human cells. J Biol Chem 279, 20067-20075
44. Frum, R. A., Chastain, P. D., 2nd, Qu, P., Cohen, S. M., and Kaufman, D. G. (2008) DNA replication in early S phase pauses near newly activated origins. Cell Cycle 7, 1440-1448
45. Chastain, P. D., 2nd, Heffernan, T. P., Nevis, K. R., Lin, L. Kaufmann, W. K., Kaufman, D. G., and Cordeiro-Stone, M. (2006) Checkpoint regulation of replication dynamics in UV-irradiated human cells. Cell Cycle 5, 2160-2167
46. Frum, R. A., Khondker, Z. S., and Kaufman, D. G. (2009) Temporal differences in DNA replication during the S phase using single fiber analysis of normal human fibroblasts and glioblastoma T98G cells. Cell Cycle 8, 3133-3148
47. Paules, R. S., Cordeiro-Stone, M., Mass, M. J., Poirier, M. C., Yuspa, S. H., and Kaufman, D. G. (1988) Benzo[alpha]pyrene diol epoxide I binds to DNA at replication forks. Proc Natl Aced Sci USA 85, 2176-2180
48. Yang, N., Galick, H., and Wallace, S. S. (2004) Attempted base excision repair of ionizing radiation damage in human lymphoblastoid cells produces lethal and mutagenic double strand breaks. DNA Repair (Amst) 3, 1323-1334
49. Tian, K., McTigue, M., and de los Santos, C. (2002) Sorting the consequences of ionizing radiation: processing of 8-oxoguanine/abasic site lesions. DNA Repair (Amst) 1, 1039-1049
50. Georgakilas, A. G., Bennett, P. V., and Sutherland, B. M. (2002) High efficiency detection of bi-stranded abasic clusters in gamma-irradiated DNA by putrescine. Nucleic Acids Res 30, 2800-2808

### REFERENCES FOR EXAMPLE 7

1. Allard, W. J., Matera, J., Miller, M. C., Repollet, M., Connelly, M. C., Rao, C., Tibbe, A. G., Uhr, J. W. and Terstappen, L. W. (2004) Tumor cells circulate in the peripheral blood of all major carcinomas but not in healthy subjects or patients with nonmalignant diseases. Clinical cancer research: an official journal of the American Association for Cancer Research, 10, 6897-6904.
2. Nakagawa, T., Martinez, S. R., Goto, Y., Koyanagi, K., Kitago, M., Shingai, T., Elashoff, D. A., Ye, X., Singer, F. R., Giuliano, A. E. et al. (2007) Detection of circulating tumor cells in early-stage breast cancer metastasis to axillary lymph nodes. Clinical cancer research: an official journal of the American Association for Cancer Research, 13, 4105-4110,
3. Wang, L. H., Pfister, T. D., Parchment, R. E., Kummar, S., Rubinstein, L. Evrard, Y. A., Gutierrez, M. E., Murgo, A. J., Tomaszewski, J. E., Doroshow, J. H. et al. (2010) Monitoring drug-induced gammaH2AX as a pharmacodynamic biomarker in individual circulating tumor cells. Clin Cancer Res, 16, 1073-1084.
4. Pachmann, K., Camara, O., Kavallaris, A., Krauspe, S., Malarski, N., Gajda, M., Kroll, T., Jorke, C., Hammer, U., Altendorf-Hofmann, A. et al. (2008) Monitoring the response of circulating epithelial tumor cells to adjuvant chemotherapy in breast cancer allows detection of patients at risk of early relapse. J Clin Oncol, 26, 1208-1215.
5. Smerage, J. B., Hayes, D. F., Doyle, G. V., Terstappen, L. W., Brown, M. E. and Schott, A. F. Journal of Clinical Oncology, 2006 ASCO Annual Meeting Proceedings Part I. Vol 24, No. 18S (June 20 Supplement), 2006: 10079.
6. Yang, L. Lang, J. C., Balasubramanian, P., Jatana, K. R., Schuller, D., Agrawal, A., Zborowski, M. and Chalmers, J. J. (2009) Optimization of an enrichment process for circulating tumor cells from the blood of head and neck cancer patients through depletion of normal cells. Biotechnol Bioeng, 102, 521-534,
7. Hou, J., Krebs, M. G., Ward, T., Morris, K., Sloane, R., Blackhall, F. H. and Dive, C. (2010) Circulating Tumor Cells, Enumeration and Beyond. Cancers, 2.
8. Al-Hajj, M., Wicha, M. S., Benito-Hernandez, A., Morrison, S. J. and Clarke, (2003) Prospective identification of tumorigenic breast cancer cells. Proc Natl Acad Sci USA, 100, 3983-3988.
9. Blick, T., Hugo, H., Widodo, E., Waltham, M., Pinto, C., Mani, S. A., Weinberg, R. A., Neve, R. M., Lenburg, M. E. and Thompson, E. W. Epithelial mesenchymal transition traits in human breast cancer cell lines parallel the CD44(hi/)CD24 (lo/−) stem cell phenotype in human breast cancer. J Mammary Gland Biol Neoplasia, 15, 235-252.
10. Blick, T., Widodo, E. Hugo, H., Waltham, M., Lenburg, M. E., Neve, R. M. and Thompson, E. W. (2008) Epithelial mesenchymal transition traits in human breast cancer cell lines. Clin Exp Metastasis, 25, 629-642.
11. Singh, A. and Settleman, J. EMT, cancer stem cells and drug resistance: an emerging axis of evil in the war on cancer. Oncogene, 29, 4741-4751.
12. Bosanquet, A. G. and Bell, P. B. (1996) Novel ex vivo analysis of nonclassical, pleiotropic drug resistance and collateral sensitivity induced by therapy provides a rationale for treatment strategies in chronic lymphocytic leukemia. Blood, 87, 1962-1971.
13. Chastain II, P., Nakamura, J, Rao, S., Chu, H., Ibrahim, J G, Swenberg, J A, and Kaufman, D G. (2010) Abasic sites preferentially form at regions undergoing DNA replication. Faseb J.
14. Chastain, P. D., 2nd, Nakamura, J., Swenberg, J. and Kaufman, D, (2006) Nonrandom AP site distribution in highly proliferative cells. FASEB J, 20, 2612-2614.
15. Talasaz, Powell, A. A., Huber, D. E., Berbee, J. G., Roh, H. H., Yu, W., Xiao, W., Davis, M. M., Pease, R. F., Mindrinos, M. N. et al. (2009) Isolating highly enriched populations of circulating epithelial cells and other rare cells from blood using a magnetic sweeper device, Proc Natl Acad Sci USA, 106, 3970-3975.
16. Amrein, L., Loignon, M., Goulet, A. C., Dunn, M., Jean-Claude, B., Aloyz, R. and Panasci, L. (2007) Chlorambucil cytotoxicity in malignant B lymphocytes is synergistically increased by 2-(morpholin-4-yl)-benzo[h]chomen-4-one (NU7026)-mediated inhibition of DNA double-strand break repair via inhibition of DNA-dependent protein kinase. J Pharmacol Exp Ther, 321, 848-855,
17. Bera, S., Greiner, S., Choudhury, A., Dispenzieri, A., Spitz, D. R., Russell, S. J. and Goel, A. Dexamethasone-induced oxidative stress enhances myeloma cell radiosensitization while sparing normal bone marrow hematopoiesis. Neoplasia, 12, 980-992.
18. Bromidge, T. J., Howe, D. J., Johnson, S. A. and Phillips, M. J. (1995) Adaptation of the TdT assay for semi-quantitative flow cytometric detection of DNA strand breaks. Cytometry, 20, 257-260.
19. Celik, H. and Arinc, E. (2008) Bioreduction of idarubicin and formation of ROS responsible for DNA cleavage by NADPH-cytochrome P450 reductase and its potential role in the antitumor effect. Pharm Pharm Sci, 11, 68-82.
20. Ewald, B., Sampath, D. and Plunkett, W. (2007) H2AX phosphorylation marks gemcitabine-induced stalled replication forks and their collapse upon S-phase checkpoint abrogation. Mol Cancer Ther, 6, 1239-1248.
21, Fung, H. and Demple, B. Distinct roles of Ape1 protein in the repair of DNA damage induced by ionizing radiation or bleomycin. J Biol Chem, 286, 4968-4977.
22. Huang, C. H., Mirabelli, O. K., Jan, Y. and Crooke, S. T. (1981) Single-strand and double-strand deoxyribonucleic acid breaks produced by several bleomycin analogues. Biochemistry, 20, 233-238.
23. Johnston, J. B. Mechanism of action of pentostatin and cladribine in hairy cell leukemia. Leuk Lymphoma, 52 Suppl 2, 43-45.
24. Minotti, G., Menna, P., Salvatorelli, E., Cairo, G. and Gianni, L. (2004) Anthracyclines: molecular advances and pharmacologic developments in antitumor activity and cardiotoxicity. Pharmacol Rev, 56, 185-229.
25. Pratibha, R. K. (2009) Enzymatic Studies of Etoposide-Induced Oxidative Stress in Hepatic Tissue of Rats. The Free Library.
26. Ramanathan, B., Jan, K. Y., Chen, C. H., Hour, T. C., Yu, H. J. and Pu, Y. S. (2005) Resistance to paclitaxel is proportional to cellular total antioxidant capacity. Cancer Res, 65, 8455-8460.
27. Rojas, E., Mussali, P., Tovar, E. and Valverde, M. (2009) DNA-AP sites generation by etoposide in whole blood cells. BMC Cancer, 9, 398.
28. Saris, C. P., van de Vaart, P. J., Rietbroek, R. C. and Blommaert, F. A. (1996) In vitro formation of DNA adducts by cisplatin, lobaplatin and oxaliplatin in calf thymus DNA in solution and in cultured human cells. Carcinogenesis, 17, 2763-2769.
29. Smart, D. J., Halicka, H. D., Schmuck, G., Traganos, F., Darzynkiewicz, Z. and Williams, G. M. (2008) Assessment of DNA double-strand breaks and gammaH2AX induced by the topoisomerase II poisons etoposide and mitoxantrone. Mutat Res, 641, 43-47.
30. Snell, M. R., Liu, L. and Gerson, S. L. (2004) Enhanced sensitivity to fludarabine in colon cancer cells co-treated with methoxyamine. AACR Meeting Abstracts, 2004, 346.
31. Timur, M., Akbas, S. H. and Ozben, T. (2005) The effect of Topotecan on oxidative stress in MCF-7 human breast cancer cell line. Acta Biochim Pol, 52, 897-902.
32. Tripathi, D. N. and Jena, G. B. (2008) Ebselen attenuates cyclophosphamide-induced oxidative stress and DNA damage in mice. Free Radic Res, 42, 966-977.
33. Vibet, S., Maheo, K., Gore, J., Dubois, P., Bougnoux, P. and Chourpa, I. (2007) Drfferential subcellular distribution of mitoxantrone in relation to chemosensitization in two human breast cancer cell lines. Drug Metab Dispos, 35, 822-828.
34. Vock, E. H., Lutz, W. K., Hormes, P., Hoffmann, H. D. and Vamvakas, S. (1998) Discrimination between genotoxicity and cytotoxicity in the induction of DNA double-strand breaks in cells treated with etoposide, melphalan, cisplatin, potassium cyanide, Triton X-100, and gamma-irradiation. Mutat Res, 413, 83-94.
35. Waters, T. R., Gallinari, P., Jiricny, J. and Swann, P. F. (1999) Human thymine DNA glycosylase binds to apurinic sites in DNA but is displaced by human apurinic endonuclease 1. J Biol Chem, 274, 67-74.
36. Wyatt, M. D. and Wilson, D. M., 3rd. (2009) Participation of DNA repair in the response to 5-fluorouracil. Cell Mol Life Sci, 66, 788-799.
37. Xie, C., Edwards, H., Xu, X. Zhou, H., Buck, S. A., Stout, M. L., Yu, Q. Rubnitz, J. E. Matherly, L. H., Taub, J. W. et al. Mechanisms of synergistic antileukemic interactions between valproic acid and cytarabine in pediatric acute myeloid leukemia. Clin Cancer Res, 16, 5499-5510.
38, Zecevic, A., Sampath, D., Ewald, B., Chen, R., Wierda, W. and Plunkett, W. Killing of chronic lymphocytic leukemia by the combination of fludarabine and oxaliplatin is dependent on the activity of XPF endonuclease. Clin Cancer Res, 17, 4731-4741.
39. Zheng, H., Wang, X., Legerski, R. J., Glazer, P. M. and Li, L. (2006) Repair of DNA interstrand cross-links: interactions between homology-dependent and homology-independent pathways. DNA Repair (Amst). 5, 566-574.
40, Nazarewicz, R. R., Zenebe, W. J., Parihar, A., Larson, S. K., Alidema, E., Choi, J. and Ghafourifar, P. (2007) Tamoxifen induces oxidative stress and mitochondrial apoptosis via stimulating mitochondrial nitric oxide synthase. Cancer research, 67, 1282-1290.
41. Ferlini, C., Scambia, G., Marone, M., Distefano, M., Gaggini, C., Ferrandina, G., Fattorossi, A., Isola, G., Benedetti Panici, P. and Mancuso, S. (1999) Tamoxifen induces oxidative stress and apoptosis in oestrogen receptor-negative human cancer cell lines. Br Cancer, 79, 257-263.
42, Kim, S. K., Yang, J. W., Kim, M. R., Roh, S. H., Kim, H. G., Lee, K. Y., Jeong, H. G. and Kang, K. W. (2008) Increased expression of Nrf2/ARE-dependent anti-oxidant proteins in tamoxifen-resistant breast cancer cells. Free Radic Biol Med, 45, 537-546.
43. Hair, J. M., Terzoudi, G. I., Hatzi, V. I., Lehockey, K. A., Srivastava, D., Wang, W., Pantelias, G. E. and Georgakilas, A. G. (2010) BRCA1 role in the mitigation of radiotoxicity and chromosomal instability through repair of clustered DNA lesions. Chem Biol Interact, 188, 350-358.
44. Holt, S. M., Scemama, J. L., Panayiotidis. M1 and Georgakilas, A. G. (2009) Compromised repair of clustered DNA damage in the human acute lymphoblastic leukemia MSH2-deficient NALM-6 cells. Mutat Res, 674, 123-130.
45, Francisco, D. C., Peddi, P., Hair, J. M., Flood, B. A., Cecil, A. M., Kalogerinis, P. T., Sigounas, G. and Georgakilas, A. G. (2008) Induction and processing of complex DNA damage in human breast cancer cells MCF-7 and nonmalignant MCF-10A cells. Free Radic Biol ivied, 44, 558-569.
46. Baird, B. J., Dickey, J. S., Nakamura, A. J., Redon, C. E., Parekh, P., Griko, Y. V., Aziz, K., Georgakilas, A. G., Bonner, W. M. and Martin, O. A. (2011) Hypothermia postpones DNA damage repair in irradiated cells and protects against cell killing. Mutat Res, 711, 142-149.
47. Kohen, R. and Nyska, A. (2002) Oxidation of biological systems: oxidative stress phenomena, antioxidants, redox reactions, and methods for their quantification, Toxicol Pathol, 30, 620-650.
48. Charafe-Jauffret, E., Ginestier, C., Iovino, F., Wicinski, J., Cervera, N., Finetti, P., Hur, M. H., Diebel, M. E., Monville, F., Dutcher, J. et al. (2009) Breast cancer cell lines contain functional cancer stem cells with metastatic capacity and a distinct molecular signature, Cancer Res, 69, 1302-1313.
49, Keen, J. C., and Davidson, N. E. (2003) The biology of breast carcinoma, Cancer, 97, 825-833.
50. Paridaens, R., Biganzoli, L., Bruning, P., Klijn, J. G., Gamucci, T., Houston, S., Coleman, R., Schachter, J., Van Vreckem, A., Sylvester, R. et al. (2000) Paclitaxel versus doxorubicin as first-line single-agent chemotherapy for metastatic breast cancer: a European Organization for Research and Treatment of Cancer Randomized Study with cross-over, J Clin Oncol, 18, 724-733.
51. Smith, L., Watson, M. B., O'Kane, S. L., Drew, P. J., Lind, M. J. and Cawkwell, L. (2006) The analysis of doxorubicin resistance in human breast cancer cells using antibody microarrays. Molecular cancer therapeutics, 5, 2115-2120,
52. Nakayama, S., Torikoshi, Y., Takahashi, T., Yoshida, T., Sudo, T., Matsushima, T., Kawasaki, Y., Katayama, A., Gohda, K, Hortobagyi, G. N. et al. (2009) Prediction of paclitaxel sensitivity by CDK1 and CDK2 activity in human breast cancer cells, Breast Cancer Res, 11, R12.
53, Zhong, Y., Zhang, F., Sun, Z., Zhou, W., Li, Z. Y., You, Q. D., Guo, Q. L. and Hu, R. (2012) Drug resistance associates with activation of Nrf2 in MCF-7/DOX cells, and wogonin reverses it by down-regulating Nrf2-mediated cellular defense response. Mol Carcinog.
54. Tassone, P. Tagliaferri, P., Perricelli, A., Blotta, S., Quaresima, B., Martelli, M. L., Goel, A., Barbieri, V., Costanzo, F. Boland, C. R. et al. (2003) BRCA1 expression modulates chemosensitivity of BRCA1-defective HCC1937 human breast cancer cells. Br J Cancer, 88, 1285-1291.
55. CAKABAY, B. (2011) Triple-Negative Breast Cancer: Frequency, Molecular Subtypes, and Therapeutic Options. European Journal of Surgical Sciences, 2, 57-61.
56. Song, S., Yu, B., Wei, Y., Wientjes, M. G, and Au, J. L. (2004) Low-dose suramin enhanced paclitaxel activity in chemotherapy-naive and paclitaxel-pretreated human breast xenograft tumors. Clin Cancer Res, 10, 6058-6065.
57. Newman, S. P. Foster, P. A., Stengel, C., Day, Ho, Y. T., Judde, J. G., Lassalle, M., Prevost, G., Leese, M. P., Potter, B. V. et al. (2008) STX140 is efficacious in vitro and in vivo in taxane-resistant breast carcinoma cells. Clin Cancer Res, 14, 597-606.
58. Nakayama, S., Torikoshi, Y., Takahashi, T., Yoshida, T., Sudo, T., Matsushima, T., Kawasaki, Y., Katayama, A., Gohda, K., Hortobagyi, G. N. et al. (2009) Prediction of paclitaxel sensitivity by CDK1 and CDK2 activity in human breast cancer cells. Breast Cancer Res, 11, R12.
59. Ameri, K. Luong, R., Zhang, H., Powell, A. A., Montgomery, K. D., Espinosa, I. Bouley, D. M., Harris, A. L. and Jeffrey, S. S. (2010) Circulating tumour cells demonstrate an altered response to hypoxia and an aggressive phenotype. Br J Cancer, 102, 561-569.
60. Mizutani, H., Oikawa, S., Hiraku, Y. Murata, M., Kojima, M. and Kawanishi, S. (2003) Distinct mechanisms of site-specific oxidative DNA damage by doxorubicin in the presence of copper(II) and NADPH-cytochrome P450 reductase. Cancer Sci, 94, 686-691.
61. Tassone, P., Tagliaferri, P. Perricelli, A., Blotta, S., Quaresima, B., Martelli, Goel, A., Barbieri, V., Costanzo, F., Boland, C. R. et al. (2003) BRCA1 expression modulates chemosensitivity of BRCA1-defective HCC1937 human breast cancer cells. Br J Cancer, 88, 1285-1291.
62. Gajria, D., Seidman, A. and Dang, C. (2010) Adjuvant taxanes: more to the story. Clin Breast Cancer, 10 Suppl 2, S41-49.
63. Dougherty, M. K., Schumaker, L. M., Jordan, V. C., Welshons, W. V., Curran, E. M., Ellis, M. J. and El-Ashry, D. (2004) Estrogen receptor expression and sensitivity to paclitaxel in breast cancer. Cancer Biol Ther, 3, 460-467.
64. Liu, F., Jiang, Z. F., Song, S. T., Gun. J. Z., Zhang, S. H. and Feng, S. Q. (2006) [HER-2 and ER expression in prediction of chemo-sensitivity of taxane for advanced breast cancer]. Zhonghua Zhong Liu Za Zhi, 28, 449-451.
65. Silver, D. P., Richardson, A. L., Eklund, A. C., Wang, Z. C., Szallasi, Z., Li, Q., Juul, N., Leong, C. O., Calogrias, D., Buraimoh, A. et al, (2010) Efficacy of neoadjuvant Cisplatin in triple-negative breast cancer. J Clin Oncol, 28, 1145-1153,
66. Euhus, D. M. (2011) New insights into the prevention and treatment of familial breast cancer. J Surg Oncol, 103, 294-298.
67. Orlowski, R. Z. and Dees, E. C. (2003) The role of the ubiquitination-proteasome pathway in breast cancer: applying drugs that affect the ubiquitin-proteasome pathway to the therapy of breast cancer. Breast Cancer Res, 5, 1-7.
68. Bae, I., Fan, S., Meng, Q., Rih, J. K., Kim, H. J., Kang, H. J., Xu, J., Goldberg, I. D., Jaiswal, A. K. and Rosen, E. M. (2004) BRCA1 induces antioxidant gene expression and resistance to oxidative stress, Cancer Res, 64, 7893-7909.
69. Kotsopoulos, J., Shen, H., Rao, A. V., Poll, A., Ainsworth, P., Fleshner, N. and Narod, S. A. (2008) A BRCA1 mutation is not associated with increased indicators of oxidative stress, Clin Breast Cancer, 8, 506-510,
70, Adams, A. A., Okagbare, P., Feng, J., McCarley, R. L. Murphy, M. C. and Soper, S. A. (2008) Circulating tumor cell isolation and enumeration using polymer-based microfluidics with an integrated conductivity sensor. Journal of the American Chemical Society, 130, 8633-8641.
71, Dharmasiri, U., Balamurugan, S., Adams, A. A., Okagbare, P. I., Obubuafo, A. and Soper, S. A. (2009) Highly efficient capture and enumeration of low abundance prostate cancer cells using prostate-specific membrane antigen aptamers immobilized to a polymeric microfluidic device. Electrophoresis, 30, 3289-3300.
72. Dharmasiri, U., Njoroge, S. K., Witek, M. A., Adebiyi, M. G., Karnande, J. W., Hupert, M., Barany, F. and Soper, S. A. (2011) High-Throughput Selection, Enumeration, Electrokinetic Manipulation, and Molecular Profiling of Low-Abundance Circulating Tumor Cells Using a Microfluidic System. Analytical Chemistry, 83, 2101-2109.
73. Park, D. S. W., Hupert, Witek, M. A., You, B. H., Datta, P., Guy, J., Lee, J. B., Soper, S. A., Nikitopoulos, D. E. and Murphy, M. C. (2008) A titer plate-based polymer microfluidic platform for high throughput nucleic acid purification. Biomedical Microdevices, 10, 21-33.
74. Witek, M. A., Hupert, M. L., Park, D. S. W., Fears, K., Murphy, M. C. and Soper, S. A. (2008) 96-well polycarbonate-based microfluidic titer plate for high-throughput purification of DNA and RNA, Analytical Chemistry, 80, 3483-3491.
75. Xu, Y. C., Vaidya, B., Patel, A. B., Ford, S. M., McCarley, R. L. and Soper, S. A. (2003) Solid-phase reversible immobilization in microfluidic chips for the purification of dye-labeled DNA sequencing fragments. Analytical Chemistry, 75, 2975-2984.
76. Witek, M. A., Llopis, S., Wheatley, A., McCarley, R. and Soper, S. A. (2006) Purification and Preconcentration of Genomic DNA from Whole Cell Lysates Using Photoactivated Polycarbonate (PPC) Microfluidic Chips. Nucleic Acids Research, 34, e74.
77, Wang, H., Chen, H. W., Hupert, M. L., Chen, P. C., Datta, P., Pittman, T. L., Goettert, J., Murphy, M. C., Williams, D., Barany, F. et al. (2012) Fully Integrated Thermoplastic Genosensor for the Highly Sensitive Detection and Identification of Multi-Drug-Resistant Tuberculosis. Angewandte Chemie-International Edition, 51, 4349-4353,
78. Khoja, L. Backen, A., Sloane, R., Menasce, L. Ryder, D., Krebs, M., Board, R., Clack, G., Hughes, A., Blackhall, F. et al. (2012) A pilot study to explore circulating tumour cells in pancreatic cancer as a novel biomarker. Br J Cancer, 106, 508-516.
79, Kurihara, T., Itoi, T., Sofuni, A., Fumihide, I., Tsuchiya, T., Tsuji, S., Kentaro, I., Ikeuchi, N., Tsuchida, A., Kasuya, K. et al. (2008) Detection of circulating tumor cells in patients with pancreatic cancer: A preliminary result, Journal Hepatobiliary Pancreatic Surgery, 15, 189-195.
80. Adams, A. A., Okagbare, P. I., Feng, J., Hupert, M. L., Patterson, D., Gottert, J., McCarley, Nikitopoulos, D., Murphy, M. C. and Soper, S. A. (2008) Highly efficient circulating tumor cell isolation from whole blood and label-free enumeration using polymer-based microfluidics with an integrated conductivity sensor. Journal of the American Chemical Society, 130, 8633-8641.
81. Dharmasiri, U., Njoroge, S. K., Witek, M., Adebiyi, M. G., Karnande, J. W., Hupert, M. L., Barany, F. and Soper, S. A. (2010) High-throughput selection, enumeration, electrokinetic manipulation and molecular profiling of low-abundance circulating tumor cells using a microfluidic system. Analytical Chemistry, 83, 2301-2309.
82, Gawad, S., Schild, L. and Renaud, P. (2001) Micromachined impedance spectroscopy flow cytometer for cell analysis and particle sizing. Lab Chip, 1, 76-82.
83, Hakomori, S. S. (1990) Biochemical basis of tumor-associated carbohydrate antigens; Current trends, future perspectives, and clinical applications. Immunol Allergy Clin North Am, 10, 781-802.
84, Ayodele A. Alaiya, B. F. G. A. S. L. (2000) Cancer proteomics: From identification of novel markers to creation of artifical learning models for tumor classification. Electrophoresis, 21, 1210-1217.
85. Cone, C. D. (1975) The role of surface electrical transmembrane potential in normal and malignant mitogenesis. Ann NY Acad Sci, 238, 420-435.
86, Celt A., Cipriany, B. R., Benitez, J. J. and Craighead, H. G. (2011) Single DNA Molecule Patterning for High-Throughput Epigenetic Mapping. Analytical Chemistry, 83, 8073-8077.
87. Mirsaidov, U., Timp, W., Zou, X., Dimitrov, V., Schulten, K., Feinberg, A. P, and Timp, G. (2008) Nanoelectromechanics of Methylated DNA in a Synthetic Nanopore. BioPhysical Journal: Biophysical Letters, 12, L32-L34,
88. Lim. S. F., Karpusenko, A., Sakon, J. J., Hook, J. A., Lamar, T. A. and Riehn, R. (2011) DNA methylation profiling in nanochannels. Biomicrofluidics, 5, 034106.
89. An, N., Fleming, A. M., White, H. S. and Burrows, C. J. (2012) Crown ether-electrolyte interactions permit nanopore detection of individual DNA abasic sites in single molecules. Proceedings of the National Academy of Sciences of the United States of America, 109, 11504-11509.
90. Kim, Y., Kim, K. S., Kounovsky, K. L., Chang, R., Jung, G. Y., dePablo, J. J., Jo, K. and Schwartz, D. C. (2011) Nanochannel confinement: DNA stretch approaching full contour length. Lab On a Chip, 11, 1721-1729.
91. Mannion, J. T., Reccius, C. H., Cross, J. D, and Craighead, H. G, (2006) Conformational analysis of single DNA molecules undergoing entropically induced motion in nanochannels. Biophysical Journal, 90, 4538-4545.
92. Reccius, C. H., Stavis, S. M., Mannion, Walker, L. P. and Craighead, H. G, (2008) Conformation, length, and speed measurements of electrodynamically stretched DNA in nanochannels, Biophysical Journal, 95, 273-286.
93. Liang, X. G. and Chou, S. Y. (2008) Nanogap detector inside nanofluidic channel for fast real-time label-free DNA analysis. Nano Letters, 8, 1472-1476.
94, Tsutsui, M., He, Y., Furuhashi, M., Rahong, S., Taniguchi, M. and Kawai, T. (2012) Transverse electric field dragging of DNA in a nanochannel. Scientific Reports, 2, 1-7.
95. Cao, H., Tegenfeldt, J. O., Austin, R. H. and Chou, S. Y. (2002) Gradient nanostructures for interfacing microfluidics and nanofluidics. Applied Physics Letters, 81, 3058-3060.
96. Persson, F., Fritzsche, J., Mir, K. U., Modesti, M., Westerlund, F. and Tegenfeldt, J. O. (2012) Lipid-Based Passivation in Nanofluidics, Nano Letters, 12, 2260-2265.
97. Mimnaugh, E. G. Fairchild, C. R. Fruehauf, J. P. and Sinha, B. K. (1991) Biochemical and pharmacological characterization of MCF-7 drug-sensitive and AdrR multidrug-resistant human breast tumor xenografts in athymic nude mice. Biochem Pharmacol, 42, 391-402.
98, Cristofanilli, M., Budd, G. T., Ellis, M. J., Stopeck, A., Matera, J., Miller, M. C., Reuben, J. M., Doyle, G. V., Allard, W. J., Terstappen, L. W. et al. (2004) Circulating tumor cells, disease progression, and survival in metastatic breast cancer. N Engl J Med, 351, 781-791.

1-43. (canceled)

44. A method of assessing DNA damage in a cell, the method comprising:
(a) preparing a DNA fiber from the cell;
(b) labeling the DNA fiber with a tag comprising a detectable moiety, wherein the tag comprising the detectable moiety associates with damaged DNA; and
(c) detecting the tag comprising the detectable moiety associated with damaged DNA in the DNA fiber, thereby assessing DNA damage in the cell or
(a) contacting a cell or DNA prepared therefrom with a tag comprising a detectable moiety, wherein the tag comprising the detectable moiety associates with damaged DNA;
(b) preparing a DNA fiber from the cell or DNA prepared therefrom; and
(c) detecting the tag comprising the detectable moiety associated with damaged DNA in the DNA fiber, thereby assessing DNA damage in the cell.

45. The method of claim 44, wherein the method is a method of assessing DNA damage following an event that may damage DNA or following two or more simultaneous and/or sequential events that may damage DNA.

46. The method of claim 44, wherein the DNA fiber is formed from isolated DNA and preparing the DNA fiber comprises lysing the cell, wherein the cell is maintained at an angle from about 15 to about 40 degrees from horizontal during the lysing process.

47. The method of claim 44, wherein the DNA fiber is formed from chromatin and the tag comprising the detectable moiety recognizes a protein that detects and/or repairs damaged DNA.

48. The method of claim 47, wherein the method further comprises labeling the DNA fiber with a reagent that indicates DNA replication, wherein the reagent that indicates DNA replication recognizes a replication protein and comprises a detectable moiety that is different from the detectable moiety(ies) used to label the damaged DNA.

49. The method of claim 44, wherein the method is carried out on a microscope slide.

50. The method of claim 44, wherein the cell is from a subject and the method further comprises administering to the subject a reagent that indicates DNA replication, wherein the reagent that indicates DNA replication comprises a detectable moiety that is different from the detectable moiety(ies) used to label the damaged DNA.

51. The method of claim 50, wherein the method further comprises determining whether the subject is at an elevated risk of developing a pre-cancerous or cancerous lesion, an age-related and/or chronic disorder such as ischemia/reperfusion injury, Alzheimer's disease, amylotrophic lateral sclerosis, Parkinson's disease, atherosclerosis, cataracts and/or macular degeneration.

52. The method of claim 44, wherein the method further comprises (a) contacting the DNA fiber with a test agent prior to and/or concurrently with labeling the DNA fiber with the tag comprising the detectable moiety that associates with damaged DNA; or (b) contacting the cell or DNA therefrom with a test agent prior to and/or concurrently with contacting the cell or DNA therefrom with the tag comprising the detectable moiety that associates with damaged DNA.

53. The method of claim 52, wherein the test agent is a chemical agent, an electromagnetic agent, ultraviolet radiation, ionizing radiation and/or an agent that causes oxidative damage to DNA.

54. The method of claim 44, wherein the method comprises labeling the DNA fiber with a second tag that associates with a different form of DNA damage than the first tag, and wherein the second tag comprises a second detectable moiety that differs from the first detectable moiety.

55. The method of claim 44, wherein the method further comprises labeling the DNA fiber with a reagent that associates with DNA, wherein the reagent that associates with DNA comprises a detectable moiety that is different from the detectable moiety(ies) used to label the damaged DNA.

56. The method claim 44, wherein the method further comprises labeling the DNA fiber with a reagent that indicates DNA replication, wherein the reagent that indicates DNA replication comprises a detectable moiety that is different from the detectable moiety(ies) used to label the damaged DNA.

57. The method of claim 44, wherein the method is semi-automated or is automated.

58. The method of claim 44, wherein the DNA damage comprises oxidative damage, photolesions, bulky adducts, protein-DNA crosslinks, DNA crosslinks, single-stranded DNA breaks and/or double-stranded DNA breaks.

59. The method of claim 44, wherein the type, amount and/or distribution of DNA damage is assessed.

60. The method of claim 44, wherein DNA damage within specific regions of the genome is assessed.

61. The method claim 44, wherein the tag comprising the detectable moiety is an aldehyde reactive probe that recognizes AP sites comprising a detectable moiety.

62. The method of claim 61, wherein the detectable moiety is biotin or a fluorescent moiety.

63. The method of claim 44, wherein the DNA fiber is prepared in a microfluidic or nanofluidic device and detecting the tag comprises the detectable moiety associated with damaged DNA is carried out in a microfluidic or a nanoflidic device.

64. A method of assessing DNA damage in a cell, the method comprising,
(a) preparing a DNA fiber from the cell;
(b) introducing the DNA fiber into a microfluidic or nanofluidic channel,
(c) establishing a voltage across or through the channel; and
(d) detecting a change in the electrical current across the channel as the DNA moves through the channel, thereby assessing the DNA damage in the cell.

65. The method of claim 64, wherein establishing a voltage across or through the channel comprises using at least one pair of electrodes.

66. The method of claim 64, wherein establishing a voltage across or through the channel comprises using two pairs of electrodes.

67. The method of claim 64, wherein the method further comprises determining whether the subject is at an elevated risk of developing a pre-cancerous or cancerous lesion, an age-related and/or chronic disorder such as ischemia/reperfusion injury, Alzheimer's disease, amylotrophic lateral sclerosis, Parkinson's disease, atherosclerosis, cataracts and/or macular degeneration.

68. The method of claim 64, wherein the method further comprises contacting the cell or DNA fiber therefrom with a test agent.

69. The method of claim 68, wherein the test agent is a chemical agent, a chemotherapeutic agent, an electromagnetic agent, ultraviolet radiation, ionizing radiation, a dermatological agent and/or an agent that causes oxidative damage to DNA.

70. The method of claim 64, wherein the DNA damage comprises oxidative damage, photolesions, bulky adducts, protein-DNA crosslinks, DNA crosslinks, single-stranded DNA breaks and/or double-stranded DNA breaks.

71. The method of claim 64, wherein the DNA fiber is labeled with a detectable moiety prior to or concurrently with introducing the DNA fiber into the microfluidic or nanofluidic channel, wherein the detectable moiety is an antibody or a reagent that associates with DNA.
